(19) United States
(12) Patent Application Publication
(10) Pub. No.: US 2003/0087280 A1
(43) Pub. Date: May 8, 2003

(54) Shot gun optical maps of the whole E. coli O157:H7

(76) Inventors: David C. Schwartz, Madison, WI (US); Eileen T. Dimalanta, Madison, WI (US); Sang Alex Lim, Madison, WI (US); Galex Sunyul Yen, Gurnee, IL (US); Konstantinos D. Potamousis, Madison, WI (US); Thomas S. Anantharaman, De Forest, WI (US)

Correspondence Address: DEWITT ROSS & STEVENS S.C., 8000 EXCELSIOR DR, SUITE 401, MADISON, WI, 53717-1914, US

(21) Appl. No.: 10/224,881

(22) Filed: Aug. 21, 2002

Related U.S. Application Data
(63) Continuation-in-part of application No. 09/838,497, filed on Apr. 19, 2001.
Application No. 09/838,497 is a continuation-in-part of application No. 09/175,824, filed on Oct. 20, 1998, now Pat. No. 6,221,592.

(51) Int. Cl. C12Q 1/68; G06F 19/00; G01N 33/48; G01N 33/50

(52) U.S. Cl. 435/6; 702/20

(57) ABSTRACT

Disclosed are consensus optical contig maps of an entire genome of an organism and methods of using the maps to verify, validate, refute and/or access the accuracy of a known nucleic acid sequence or full genomic sequence. The maps are constructed using optical methods wherein DNA is elongated and fixed along their length onto a solid planar surface so that the DNA molecules are individually analyzable and accessible for enzymatic reactions. The DNA molecules are then reacted with a restriction enzyme to yield cleaved fragments of discernable length fixed to the surface. The lengths of the fragments are then determined, using optical methods. From the lengths determined, an optical contig map is assembled and can be compared to a corresponding known sequence, for example, a genomic sequence.

### PRIORITY

This is a continuation-in-part of co-pending application Ser. No. 09/838,497, filed Apr. 19, 2001, which is a continuation-in-part of application Ser. No. 09/175,824, filed Oct. 20, 1998, now U.S. Pat. No. 6,221,592. The contents of each of the foregoing applications and patents is incorporated herein by reference.

### FEDERAL FUNDING

This invention was made with United States Government support awarded by the following Agency: NIH A144387. The United States has certain rights in this invention.

### BACKGROUND OF THE INVENTION

Modern approaches to understanding the detailed molecular mechanisms that underline microbial biological systems often start with whole genome sequencing and annotation (Ruepp et al., 2000, Nature 407:508-513; Shigenobu et al., 2000, Nature 407:81-86; Stover et al., 2000, Nature 406:959-964). Since the first microbe was fully sequenced in 1995 (Fleischmann et al., 1995, Science 269:496-512), a large number of microbial genomes have been sequenced and an even larger number are slated to be completed in the near future. Although new sequencing technologies have to some extent ameliorated the daunting task of amassing the large number of sequence reads required to assemble a completed genome sequence, significant progress has not been made in new approaches to finish and validate these data. Whole genome shotgun sequencing techniques are widely used to eliminate the need for time-consuming mapping. However, shotgun sequencing approaches have not totally eliminated the requirement for maps. Rather, shotgun mapping requires new types of maps in order to complement fully these high-throughput approaches.

Optical mapping is an approach for constructing whole genome maps from genomic DNA molecules directly extracted from both bacteria and unicellular parasites. See Lai et al., 1999, Nat. Genet. 23:309-313; and Lin et al., 1999, Science 285:1558-1562. Optical mapping creates ordered restriction maps using randomly selected individual DNA molecules mounted on specially prepared surfaces (Aston et al., 1999, Trends Biotechnol. 17:287-302; Aston et al., 1999, Methods Enzymol. 303:55-73; and Jing et al., 1999, Genome Res. 9:175-181; see also Lai et al., 1999 and Lin et al., 1999, supra). See also U.S. Pat. Nos. 6,294,136; 6,221,592; 6,174,671, 6,150,089; 5,720,928; 5,599,664; and 5,405,519. The approach does not require electrophoresis, hybridization, PCR, or cloning. Ordered restriction maps of an entire genome form a useful scaffold for guiding sequence assembly and for validating finished sequence. Because such maps are directly linked with the genome, they do not suffer from clone- or PCR-based artifacts. This makes them ideal for cross-checking sequencing efforts. Previous whole genome optical maps have served in this capacity to aid large-scale sequencing efforts. See Lai et al., 1999; and Lin et al., 1999, supra.

Pathogenic microbes are numerous and clinically important. Often however they are lacking well-developed genomic resources such as genetic markers, simple physical maps, and definitively-characterized genome structural features. Such organisms are a challenge to researchers engaged in large-scale sequencing projects. This is because simple facts, such as accurate genome size and chromosome number are obscure. For example, variations in pathogenicity observed between related bacterial strains can sometimes be associated with significant alterations to genome structure (Karaolis et al., 1994, J. Clin. Microbiol. 32:796-802 and Sokurenko et al., 1998, Proc. Natl. Acad. Sci. 95:8922-8926).

Single-molecule DNA analysis is a subset of physical and genetic mapping that has been applied to mapping and cloning of disease genes and to direct sequencing efforts. Known methods of visualizing single DNA molecules include fluorescence microscopy in solution, fluorescence in situ hybridization (FISH), visualization by scanning tunneling microscopy or atomic force microscopy techniques, visualization of circular DNA molecules, DNA bending in transcription complexes by scanning force microscopy, direct mechanical measurement of the elasticity of single DNA molecules using magnetic beads, alignment and detection of DNA molecules involving either elongation of end-tethered surface-bound molecules by areceding air-water interface (see U.S. Pat. Nos. 5,079,169 and 5,380,833), and elongation of non-tethered molecules by “fluid fixation” (see U.S. Pat. No. 6,147,198).

In the approaches where nucleic acid molecules have been immobilized on a surface, they must then be imaged and analyzed. Although the spatial resolution of conventional light microscopy is limited, cooled, charged-coupled imaging devices (CCDs) have stimulated the development of new optical approaches to quantifying nucleic acid phenomena. The resolution of these approaches is improving to the point that they may supplant electrophoresis-based techniques in many applications. For example, Yanagida and coworkers (Yanagida et al., 1996, in “Applications of fluorescence in the biomedical sciences,” Taylor et al. (eds), Alan Liss, New York, pp. 321-345) first investigated the molecular motions of fluorescently stained individual DNA molecules in solution by image-enhanced fluorescence microscopy. Optical mapping was subsequently developed for the rapid production of ordered restriction maps from individual, fluorescently-stained DNA molecules (Cai et al., 1995, Proc. Natl. Acad. Sci. USA 92:5164-5168; Meng et al., 1995, Nature Genet. 9:432-438; Wang et al., 1995, Proc. Natl. Acad. Sci. USA 92:165-169; Schwartz et al., 1993, Science 262:110-114; Schwartz et al., 1997, Curr. Opinions in Biotechnology 8:70-74; Samad et al., Nature 378:516-517; and Samad et al., 1995, Genomic Research 59:1-4).

In the original optical mapping method, individual fluorescently-labeled yeast chromosomes were elongated and fixed in a flow of molten agarose generated between a coverslip and a glass slide. After activating an added restriction enzyme, cleavage events were recorded as time-lapse images. Cleavage sites appeared as growing gaps due to relaxation of DNA coils at nascent ends. Maps were then constructed by measuring fragment sizes using relative fluorescent intensity or apparent length measurements.

In another approach, DNA molecules (2 kb to 1,500 kb) were elongated and fixed using the flow and adhesion forces generated when a fluid sample was compressed between two glass surfaces, one of which was derivatized with polylysine or APTES (see Cai et al., 1995, supra). The fixed molecules were then digested with one or more restriction enzymes, fluorescently stained, and optically mapped.

The two approaches just described, however, provide only limited access to the samples and cannot readily accommodate arrayed samples.

Single molecule tethering techniques, as listed above, generally involve individual nucleic acid molecules that have been immobilized onto a surface via one or both of their ends. The molecules have then been further manipulated such that the molecules are stretched out. These techniques, while functional, are not ideally suited to genomic analysis. First, the required steps involved are time-consuming and can only be accomplished with a small number of molecules per procedure. Second, as a general proposition, the tethered molecules cannot be stored and used again.

The recently published human genome maps provide the first rough draft of the human DNA genome. Venter et al., 2001, Science 291:1304-1350; International Human Genome Sequencing Consortium, 2001, Nature 409:860-921. The achievement of this important milestone in genomic science was made possible through a combination of technological and organizational breakthroughs. The human genome map is poised to serve as the touchstone for major discoveries in the biological sciences. Clearly, however, deciphering the nucleotide sequence of the human genome is only the first and perhaps smallest step in the process. The challenge at hand is to discern the biological and biochemical significance embedded within the roughly 3,000,000,000 bases within the human genome. The same problem exists in the study of other, less massive genomes, such as those of bacteria and virus. Careful and comprehensive study of transcriptional patterns within different organisms, cell-types, and environments is a critical consideration in this effort. Underlying these studies are the basic biochemical mechanisms that define transcriptional activities at the molecular level.

Clearly then, a map accurately reflecting the genomic structure and sequence of E. coli 0157:H7 would be very beneficial in formulating diagnostic approaches to detecting E. coli in foodstuffs, and in developing treatment regimes to prevent E. coli infection or to ameliorate the course of the infection once begun.

### SUMMARY OF THE INVENTION

Disclosed and claimed herein is an optically-generated map of the entire genome for E. coli 0157:H7 EDL933. This particular species and strain of E. coli produces a Shiga toxin that causes over 100,000 cases of human illness annually in the United States alone. This particular strain of E. coli also poses a significant threat to public health worldwide. Over 85% of the E. coli infections in the United States are linked to contaminated food (Mead et al., 1999, Emerg. Infect. Dis. 5:607-625).

To sequence and annotate the virulant E. coli 0157:H7 bacterium, the Blattner laboratory adopted a strategy of using the E. coli K-12 genome (Blattner et al., 1997, Science 277:1453-1462) as a backbone for new sequence assembly and annotation. This strategy was designed to highlight a subset of additional candidate genes for further characterization by comparison of the 0157:H7 sequence to that of the nonpathogenic E. coli K-12. The 0157:H7 genome was expected to be considerably larger than that of the K-12, based on the sizes of fragments generated by digestion of genomic DNA with a rare cutting restriction enzyme (Bergthorsson et al., 1998, Mol. Biol. Evol. 15:6-16). However those regions common to both genomes were expected to be nearly identical (Whittam et al., 1998, Emerg. Infect. Dis. 4:615-617). Conventional genome sequencing has now confirmed that there are extensive differences between the two genomes. Furthermore these differences are distributed throughout a backbone of highly conserved and basically colinear shared genes (Blattner et al., 1997, supra; Perna et al., 2001, Nature 409:529-533).

A strategy employed in the 0157:H7 genome project was to capitalize on this backbone by using sequences similar to the regions of the K-12 genome as an indicator of contig order and to direct gap closure. The optical maps disclosed herein were undertaken to provide a unique scaffold for assembly of the 0157:H7 genome. The optical maps, however, are also useful in that they provide an invaluable early indication of major genomic rearrangements. Early indications of such major gaps greatly simplify the efforts required to close those gaps. Thus, the subject approach is quite useful when sequencing a new organism that is closely related to an organism whose genome was previously sequenced.

More specifically, a first embodiment of the invention is directed to a method of validating or refuting a known nucleic acid sequence. Here, the method comprises first elongating and fixing a plurality of nucleic acid molecules along their length onto a solid planar surface so that the nucleic acid molecules are individually analyzable and accessible for enzymatic reactions. Then, the nucleic acid molecules are reacted with a restriction endonuclease under conditions and for a time wherein the restriction endonuclease cleaves the nucleic acid molecules at a plurality of sequence-specific positions. This results in a plurality of cleaved fragments of discernable length fixed to the surface. The lengths of the cleaved fragments so generated are determined. From the fragment lengths discerned, an optical contig map is assembled. The optical contig map is then compared to the known nucleic acid sequence, whereby the known nucleic acid sequence is validated or refuted.

This approach can be reiterated multiple times, using different restriction enzymes, to generate a series of maps depicting different sequence-specific cleavage events. These maps can then be combined into a unified optical map.

A second embodiment of the invention is specifically directed to genome-wide analsyis. Here, the invention is drawn to a method of assessing the accuracy of a known, genome-wide nucleic acid sequence of an organism. In the same fashion as the first embodiment, the method comprises: first, to individual genomic DNA molecules comprising an entire genome of the organism, the DNA molecules being elongated and fixed along their length onto a solid planar surface so that the nucleic acid molecules are individually analyzable and accessible for enzymatic reactions, reacting the DNA molecules with at least one restriction endonuclease under conditions and for a time wherein the restriction endonuclease cleaves the DNA molecules at a plurality of sequence-specific positions, thereby yielding a plurality of cleaved fragments of discernable length fixed to the surface. The lengths of the cleaved fragments so generated are then determined. From the lengths determined in the previous step, an optical contig map is assembled. The optical contig map is then compared to the known, genome-wide nucleic acid sequence, whereby the accuracy of the known nucleic acid sequence is assessed.

A third embodiment of the invention is directed to consensus optical contig maps of an entire genome of an organism, the map being constructed by the series of steps described herein.

### BRIEF DESCRIPTION OF THE FIGURES

FIG. 1 is a flow chart and images depicting optical shotgun mapping of genomic DNA. High-molecular weight DNA is extracted from cells and deposited onto an optical mapping surface. After restriction enzyme digestion, and staining with a fluorescent dye, individual molecules are imaged. Images are collected semi-automatically. The computer program Semi-Autovis is then used to convert image data into map files after a user selects the desired molecules. Maps are automatically “contiged” using the Gentig program and the results displayed using the ConVEx program. The finished map is presented as a circular chromosome using software from DNAStar.

FIG. 2A is a digital fluorescence micrograph of a typical genomic DNA molecule obtained from E. coli 0157:H7, after digestion with XhoI. The image was constructed by tiling of series of 63×images using Gencol. Co-mounted lamda bacteriophage DNA was used as a sizing standard and to estimate cutting efficiency.

FIG. 2B is a whole genome XhoI restriction map generated via shotgun optical mapping, using the digital fluorescence micrograph depicted in FIG. 2A. The outer circle represents an in silico XhoI digest of the known sequence. The second outermost circle shows the consensus optical map. The inner circles represent the individual molecule maps from which the consensus map was generated. The regions designated by white triangles are discrepancies that are detailed in Table 1.

FIG. 3 is a graph depicting XhoI digst fragment sizing results for E. coli 0157:H7 plotted versus known sequence data. The diagonal line is for reference. The error bars represent the standard deviation of the fragment sizes. Inset: Fragment sizes <10 kb.

FIG. 4 depicts an alignment of optical mapping data obtained according to the present invention versus known sequence data for E. coli 0157:H7. This is a composite optical map generated by normalizing the single-enzyme digestion maps for NheI and XhoI. The resulting multi-enzyme map was aligned with the digestion map predicted from the known genomic sequence (obtained by conventional means and computed in silico). The thick black line at the bottom of the figure represents a missing region in the NheI optical map. The arrows show discrepancies between known sequence data and the optical maps. These discrepancies correspond to those in FIG. 2B.

FIG. 5 is an illustration of the biochemical scheme for Optical Sequencing showing the series of biochemical cycles and intermittent washes.

FIG. 6 illustrates in a block-diagram form a preferred embodiment of the method of making a restriction map of the present invention.

FIG. 7 illustrates a statistical model of cuts of nucleic acid molecules.

FIG. 8 illustrates an example of the alignment detection.

FIG. 9 is a variable block diagonal matrix for the dynamic programming.

FIG. 10 illustrates in a block-diagram form a preferred embodiment of the method for searching for an optimal solution of the present invention.

### DETAILED DESCRIPTION

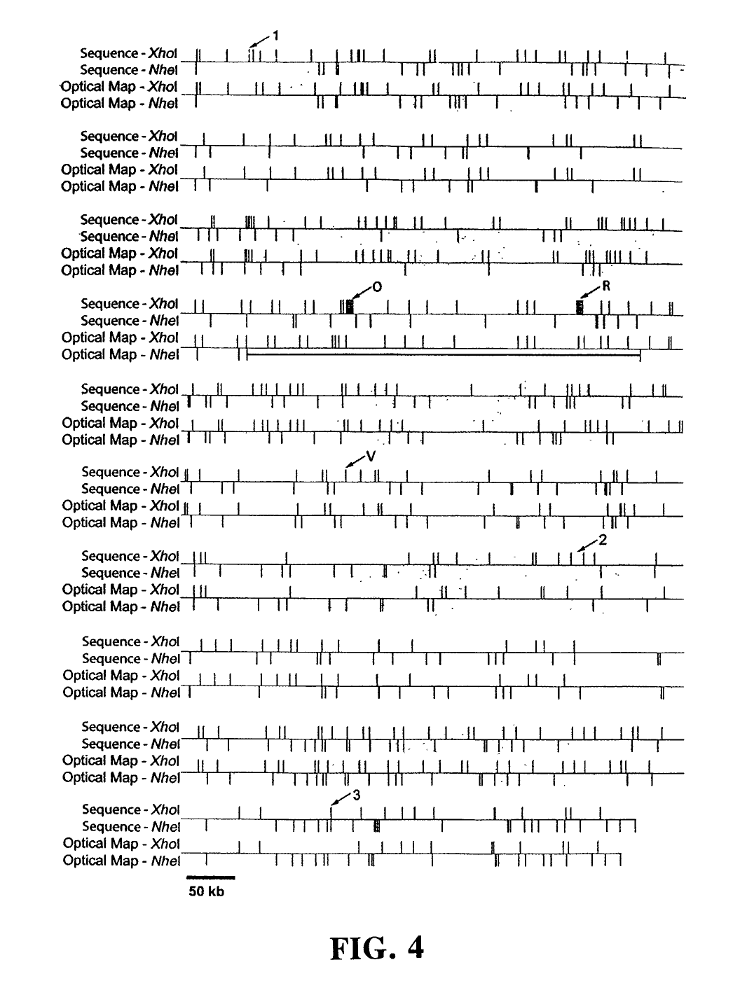

Shotgun optical mapping provides a completely independent means to validate nucleic acid sequence assemblies that does not rely on the analysis of clones. This advantage creates a direct route to sequence information that obviates artifacts created by the cloning process, which include underrepresentation of difficult regions and insert rearrangements. Although Souther blotting analysis also directly analyzes genomic DNA, it is cumbersome and difficult ro employ for high-resolution whole genome analysis. Map construction can be influenced by the use of sequencing data, so that finished maps would not represent truly independent results. To minimize any bias in sequence assembly, optical maps were constructed without detailed prior knowledge of sequence data. However, preliminary assessment of enzyme site frequencies facilitates the choice of appropriate mapping enzymes. Restriction enzymes that cut too frequently (fragments of <15 kb on the average) or too infrequently (fragments of >55 kb on the average) are not suitable for optical mapping of bacterial genomes. Problems in map assembly arise with frequent cutters because the average fragment size approaches the optical sizing error, while infrequent cutters provide sufficient information per molecule to allow confident map assemblies. To deal with these issues, partial sequence data were used to determine the approximate frequency of restriction enzyme cleavage. We transmitted the preliminary NheI map to the Blattner laboratory while they were in the early stage of sequence finishing and contig closure. At that point we determined that a critical region was not represented by the NheI map. Furthermore, it was not clear whether this region was absent or if the preliminary sequence assemblies were incorrect. Further analysis by the Blattner laboratory indicated that an XhoI map would facilitated sequence assembly efforts in this particular region (subsequently found missing in the NheI map; FIG. 4). More importantly, an NheI map would show insufficient detail to aid closure; hence an XhoI map was constructed. Given these results, future maps might be constructed in two stages; first, a “generic” optical map would be prepared in the absence of significant sequence data, later followed by an additional map (using a different enzyme) to fully leverage preliminary contig closure efforts.

Optical maps can be used to cross-check data—both derived from sequencing and other maps. Composite maps created using different enzymes require good registration to minimize errors in the relative placement of cleavage sites and thus need a way to anchor one map against another. Here, we used sequence information for this purpose, and the resulting composite map revealed discrepancies in both map and sequence data. A previous approach used in infrequent cutter to generate large fragments (in a tube) that were optically mapped (on surfaces) with a frequent cutter (Lin et al. 1999). Generally, when two maps contradict sequencing results in the same region, it is unlikely that the composite map date are incorrect. Overall, since composite maps are more informative than single enzyme maps, genomic structural details become more apparent, and these maps are a better scaffold for sequence assembly. The maps presented here were useful to the Blattner laboratory through gap closure stages by identifying errors in preliminary assemblies and characterizing contig order and gap sizes. In addition, an accurate measure of genome size is valuable for estimating the quantity of random sequence to collect before starting gap closure.

Clearly, more maps provide more useful information, but the real net utility must bejudged in a fiduciary manner as mapping versus sequence finishing costs. This equation will be different for each bacterial genome, and will depend on factors such as map resolution, as well as the nature and scope of sequencing problems. It is worthwhile considering that although the NheI map was missing a genomic region, the rest of the map was quite accurate and did greatly facilitate contig ordering. Development of a much higher through-put optical mapping system is currently underway via increasee automation and new software approaches to better link map data with sequence data. The XhoI map presented here took two weeks to complete and required the intensive effort of five individuals to prepare surfaces and mounts and edit assemblies. An important step in this direction was the development of new machine vision approaches embodied in Semi-Autovis. Recent, unpublished, developments in the optical mapping system use new surface modalities that obviate operator intervention and potentiate the ability of the machine vision to correctly identify objects for the creation o large data files. This combination would allow for a dramatic reduction in costs and woudl furhter accelerate sequence finishing efforts, as well as provide a reliable means for validation.

Strategy for mapping:

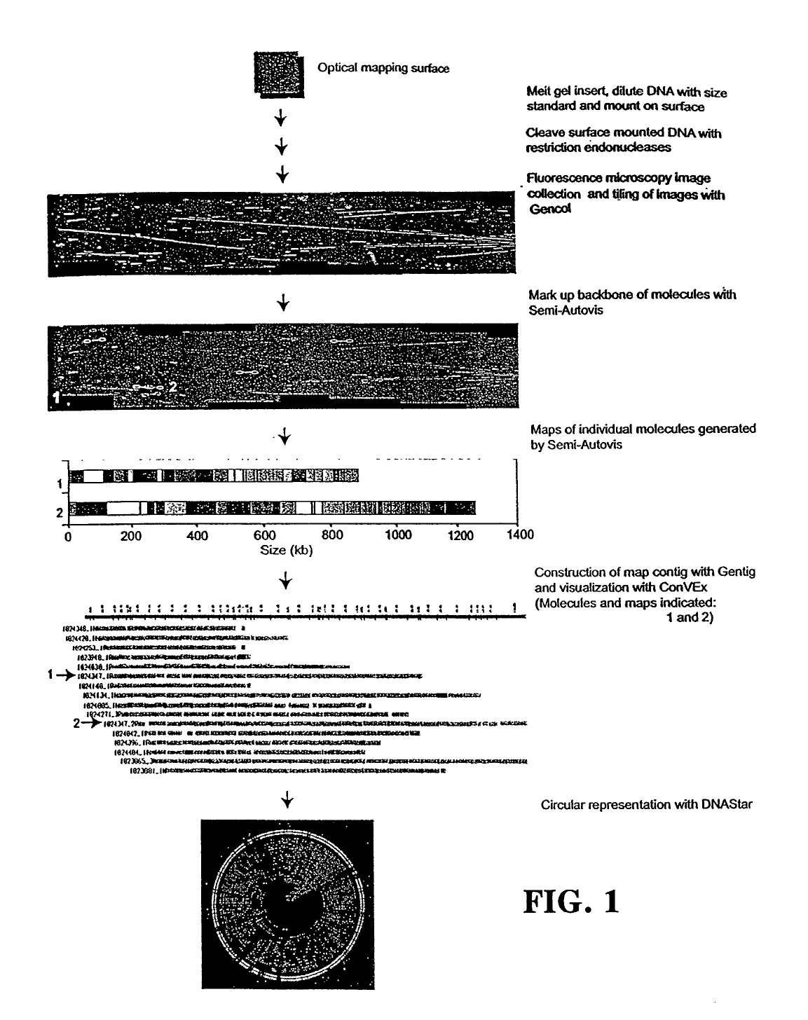

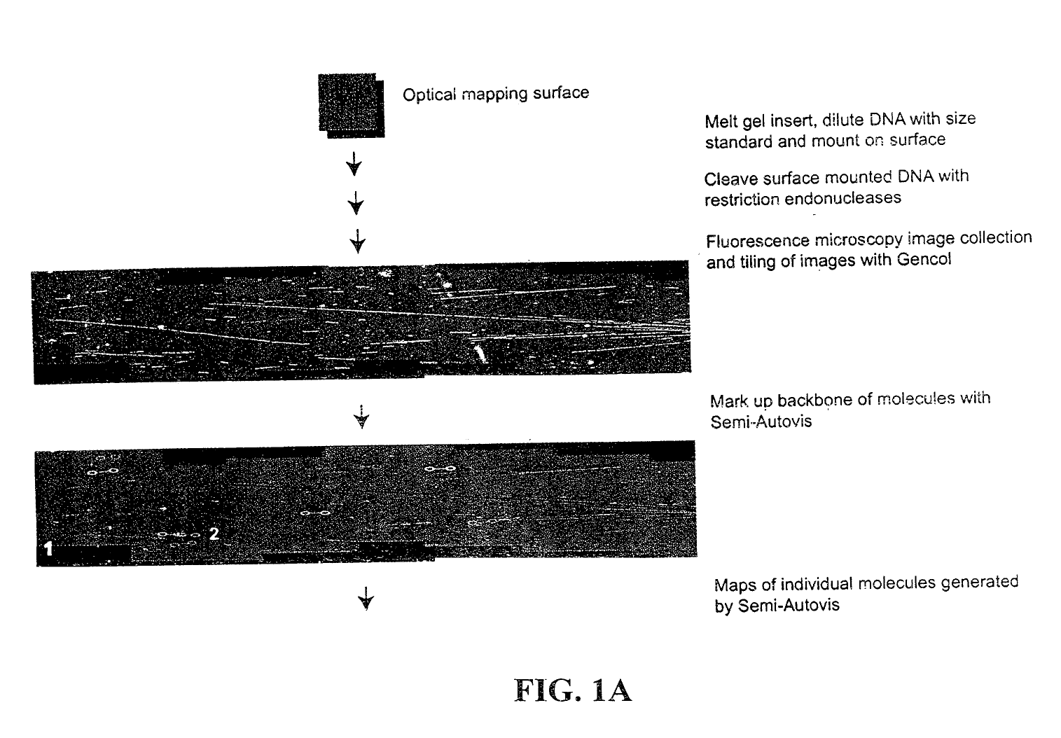

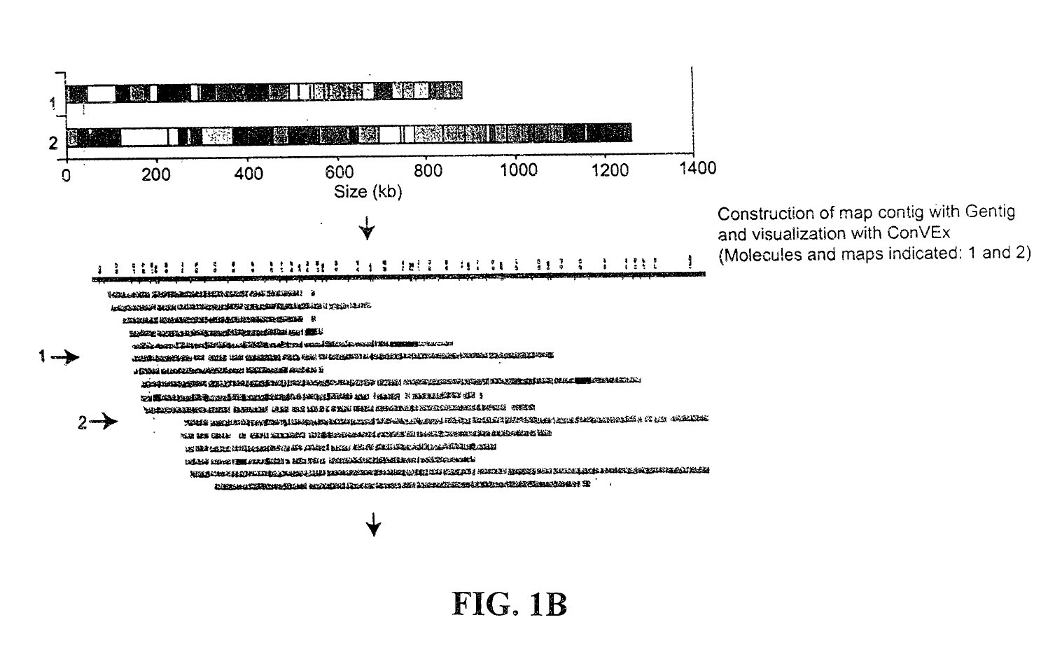

An approach to mapping entire genomes, termed shotgun optical mapping has been developed previously. See Lai et al., 1999 and Lin et al. 1999, supra. A flow chart of the approach is presented in FIG. 1. See also the Examples below for a more detailed description of how the images in FIG. 1 were assembled. Randomly broken DNA molecules that ranged in size from 150 to 219 kb were used as the mapping substrate. Molecule breakage is not deliberate but occurs as a consequence of handling. Surface-mounted molecules were digested (on an optical mapping surface, usually modified glass surfaces) with restriction endonucleases and images collected using Gencol software (see the Examples). The basis upon which shotgun optical mapping assembles whole genome maps is similar in many ways to random clone mapping approaches. In short, tiled paths can be assembled across chromosomes and the entire genome. See, for example, Marra et al., 1997, Genome Res. 7:1072-1084; Marra et al., 1997, Genome Res. 7:1072-1084; and Han et al., 2000, Genome Res. 10:714-721. In the present approach, a single molecule optical map corresponds to a cloned map discerned by gel electrophoresis. The assembly of optical maps into complete contigs covering the entire genome is a accomplished using a software program called Gentig (see Anantharaman et al., 1997, J. Comput. Biol. 4:91-118; and Lai et al., 1999, supra). The Gentig algorithms were designed to account for the types of errors unique to the analysis of single DNA maps. Error processes, such as partial digestion, spurious cuts, chimeric molecules (an imaging artifact caused by overlapping molecules), and fragment sizing errors are integrated into the Gentig program.

Optical Map of E. coli Genome:

Gentig was used to assemble two separate optical maps of E. coli 0157:H7 using two different restriction endonucleases: XhoI and NheI. The NheI map was first constructed and represents a preliminary map in that final editing was not completed. Because a long sequence stretch was not addressed by the preliminary NheI map, it became apparent that a second enzyme map was necessary. An in silico analysis of the available sequence for this organism showed that a ZhoI map would be more useful for finishing the sequence data. Additional sequence data and the ZhoI map subsequently showed that this difficult stretch (about 450 kilobases) was indeed absent from the preliminary NheI map.

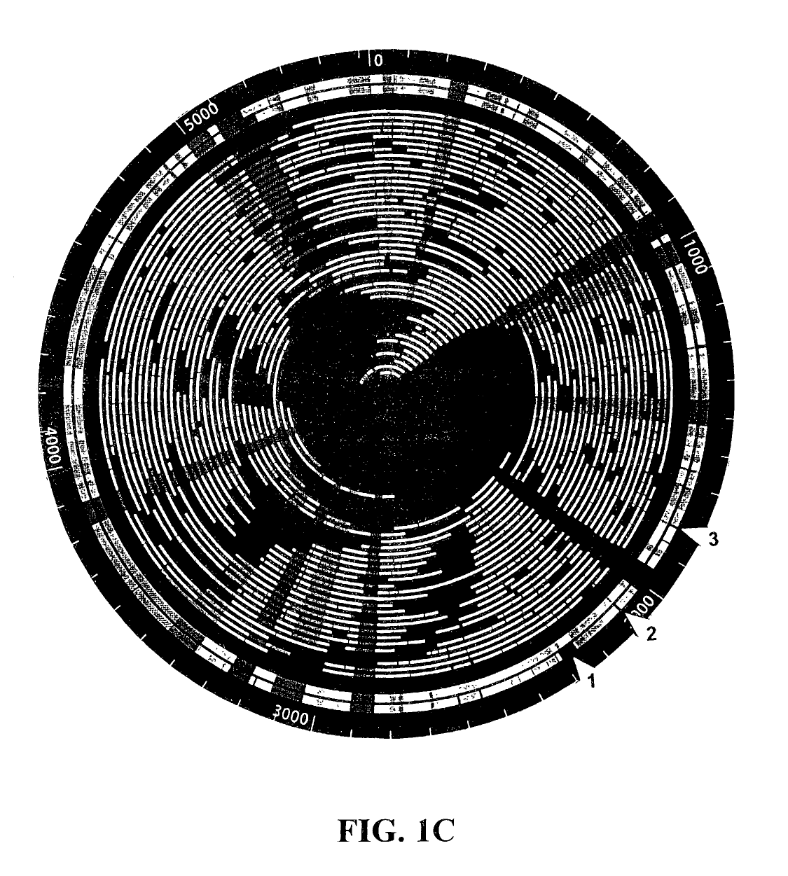

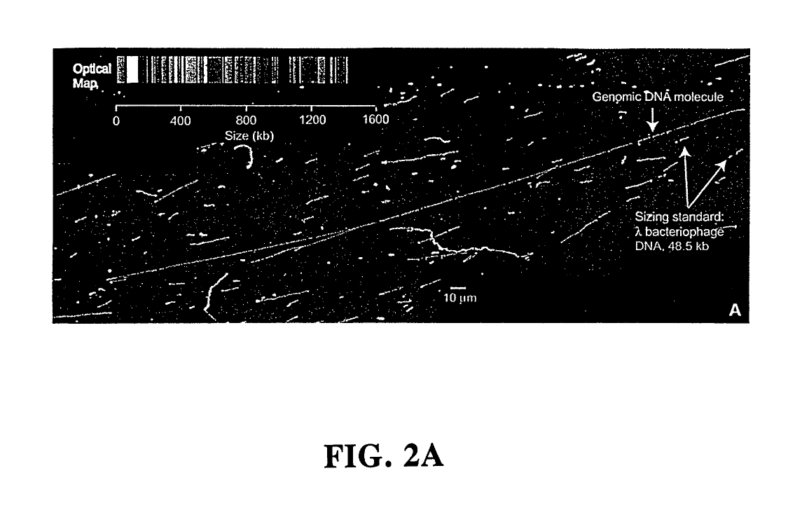

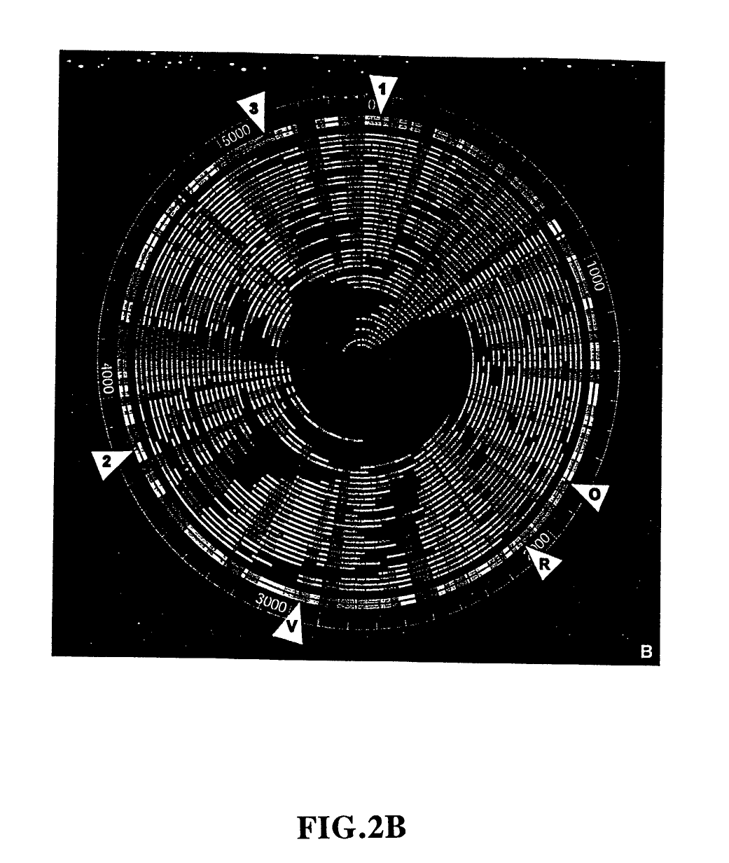

FIG. 2A shows a typical DNA molecule and its associated optical map. A total of 840 molecules were collected and processed for map construction. (ZhoI: 494 molecules collected, 251 of which went into the final contig; NheI: 346 molecules collected, 220 of which went into the final contig). The two enzymes apparently cleaved the genome to produce random patterns, with no obvious discernable structural features. However, the average fragment size differed significantly. The ZhoI map featured an average restriction fragment size of 25.1 kb versus 32.3 kb calculated for NheI.

FIG. 2B shows the finished ZhoI map constructed using Gentig and 251 molecules, thus providing 30× coverage (166 Mb of total DNA analyzed). This map formed a closed circle, with no gaps and a typical restriction fragment was computed from the average of 20 molecules. Importantly, this depth of coverage ensured confidence in calling restriction cleavage sites and accuracy in fragment sizing. The genome size was calculated to be 5.52 Mb.

Optical Map Versus Conventional Sequence Data:

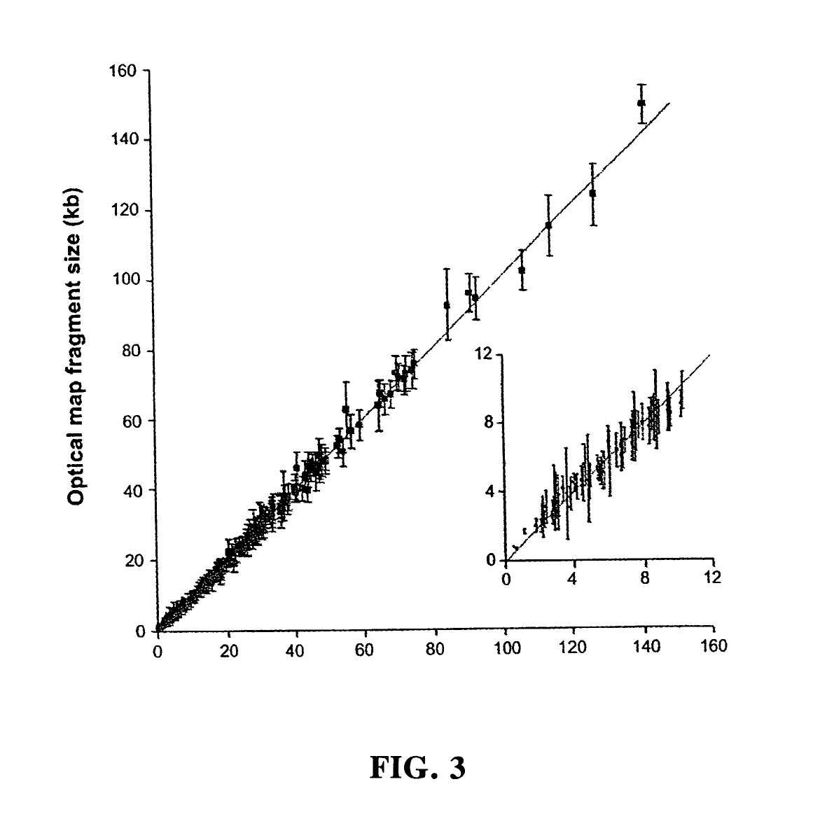

A comprehensive overview of optical mapping accuracy versus sequences shown in FIG. 3. The error bars as shown in this figure were calculated as the standard deviation on sets of homologous fragments used to calculate the average consensus map shown in FIG. 2B. Overall there was excellent agreement between map fragment sizes and those generated in silico using sequence data. For ZhoI, the precision was estimated from the median of the standard deviation determined for all fragments (2.06 kb; for a range and fragment sizes spanning 0.71 kb to 149.6 kb). The median of the absolute error (I map vs. sequence I) was 0.52 kb. Although the average percent relative error ({map/sequence}×100%) remained somewhat constant at 4.8%, the absolute error expectedly increased with fragment size.

Comparisons of the NheI optical map with the conventional sequence results showed errors similar to the ZhoI map when the missing genomic region was taken into consideration. The average and median relative errors were 5.43% and 3.32%, respectively.

Table 1 shows a detailed comparison of selected portions of the ZhoI optical map with the corresponding restriction map predicted from the conventional sequence results. These regions of the genome were selected because they show discrepancies between the optical map and the conventional sequence. Two discrepancies are readily discerned and are correspondingly noted in the Table and in FIG. 2B as “O” and “R.” These correspond to regions in the genome where there are phage insertions (CP-9330 and CP-933R, Perna et al., 2001). Manual rearrangement of some of the phage sequence here and elsewhere in the genome may result in a sequence map that aligns more closely with the optical map in these regions. The remaining discrepancies in regions 1, 2, 3, and “V” (in Table 1 and FIG. 2B) have either extra cuts in the conventional sequence or missing cuts in the optical map. The region in “V” is similar to “O” and “R” in that it contains a phage insertion (CP-933V, Perna et al., 2001). The relative error for these discrepancies was calculated by adding the sequence fragments together and comparing them to the corresponding optical map fragments.

Composite Map Constructed from Two or More Restriction Enzymes:

Composite maps constructed from multiple enzymes are more informative than a single enzyme map showing the same average fragment size (Cai et al., 1998, Proc. Natl, Acad. Sci. 95:3390-3395). For small clones, the alignment of separate maps derived from different enzymes is laborious, but straightforward. This task, howvever, becomes quite difficult when multiple map alignments must be done covering an entire genome. Previously, two separate restriction maps spanning an entire chromosome from Plasmodium falciparum were aligned (Jing et al., 1999, supra). The analysis presented therein indicated a complex set of errors, which were made apparent by local inversions in the order of closely-spaced cleavage sites between the two maps. In short, if several maps are aligned at a single end, Jing et al. revealed that the registration wanders from one end of the alignment to the other. In the present invention, the task at hand was to align two circular maps covering over 5 Mb of sequence.

TABLE 1

| | Sequence | Optical Map | | % | |
|---|---|---|---|---|---|
| | Fragment | Fragment | Difference | Relative | Standard |
| | Size (kb) | Size (kb) | (kb) | Error | Deviation |
| | 45.32 | 47.08 | −1.76 | 3.88 | 3.10 |
| | 5.99 | 7.26 | −1.27 | 21.26 | 1.93 |
| | 8.70 | 8.35 | 0.35 | 3.97 | 2.32 |
| End of one → | 15.44 | 14.00 | 1.44 | 9.36 | 2.68 |
| sequence contig | #1 | 11.73 | −11.73 | | 1.18 |
| | | 5.18 | −5.18 | | 0.55 |
| | | 9.73 | −9.73 | | 0.91 |
| Beginning of next → | 8.92 | 36.52 | −27.60 | 309.44 | 4.17 |
| sequence contig | 3.86 | 3.88 | −0.02 | 0.52 | 0.54 |
| | 28.58 | 28.20 | 0.38 | 1.32 | 2.89 |
| | 20.65 | 20.23 | 0.42 | 2.02 | 2.26 |
| | 2.79 | 2.66 | 0.12 | 4.48 | 0.37 |
| | 21.70 | 22.21 | −0.52 | 2.38 | 2.78 |
| | 25.68 | 24.60 | 1.08 | 4.19 | 3.16 |
| | 22.40 | 22.94 | −0.54 | 2.41 | 3.32 |
| | 8.88 | 8.32 | 0.56 | 6.29 | 1.34 |
| | 21.72 | 19.12 | 2.61 | 12.00 | 2.33 |
| | #2 | 6.47 | −6.47 | | 0.84 |
| | 47.68 | 49.36 | −1.68 | 3.52 | 5.29 |
| | 8.00 | 8.02 | −0.02 | 0.26 | 1.06 |
| | 10.36 | 10.39 | −0.03 | 0.27 | 1.36 |
| | 69.99 | 72.95 | −2.96 | 4.22 | 5.09 |
| | 33.68 | 35.31 | −1.63 | 4.85 | 3.47 |
| | 20.47 | 20.33 | 0.14 | 0.66 | 1.67 |
| | 24.44 | 23.54 | 0.90 | 3.68 | 2.00 |
| | 40.90 | 46.25 | −5.35 | 13.09 | 4.45 |
| | #3 | 7.95 | −7.95 | | 0.98 |
| | 0.31 | | 0.31 | | |
| | 4.02 | 4.50 | −0.49 | 12.10 | 0.73 |
| | 2.43 | 3.12 | −0.70 | 28.70 | 0.70 |
| | 0.63 | | 0.63 | | |
| | 30.84 | 28.78 | 2.05 | 6.66 | 2.17 |
| | 0.30 | | 0.30 | | |
| | 9.49 | 8.97 | 0.52 | 5.49 | 1.13 |
| | 29.53 | 28.51 | 1.02 | 3.45 | 2.76 |
| | 8.34 | 7.79 | 0.55 | 6.64 | 0.93 |
| | 24.37 | 24.57 | −0.20 | 0.83 | 2.35 |
| End of one | 10.34 | 9.88 | 0.46 | 4.47 | 1.01 |
| sequence contig → | 43.77 | 39.73 | 4.04 | 9.23 | 3.15 |
| | #4 | | | | |
| Beginning of next → | 8.65 | 7.94 | 0.71 | 8.23 | 1.27 |
| sequence contig | 21.22 | 20.77 | 0.45 | 2.14 | 2.15 |

Referring now to FIG. 4, this alignment map shows an alignment of the nascent NheI optical map with the finished XhoI optical map. The alignments were accomplished by first normalizing each map, and then breaking them into discrete sections of approximately 500 kb. Alignments were then made locally, by hand, using the in silico sequence maps as a template. Left-most alignments were done first. However, this straight forward approach does not optimally fit all restriction sites to the conventionally-generated sequence data. Errors in fragment sizing shift restriction fragments relative to each other, and this becomes apparent when large map sections are simply aligned. Statistical analysis (Jing et al., 1999) predicted that misalignment grows as the square root of the distance from a known alignment (the left end a shown in FIG. 4.) and further that smaller fragments should show more instances of position reversal. The data presented in FIG. 4 had 197 instances where consecutive restriction sites were NheI followed by XhoI (or vice-versa). In 61 of those instances the expected misalignment exceeded the distance between the restriction sites. Only half of all misalignments, on average, produce reversals of restriction site order. Hence, about 15 to 40 reversals are predicted. Actual data were observed to have 30 reversals, which is consistent with the prediction. A more appropriate approach would be to implement a set of algorithms to optimize alignments for all fragments which will rigorously model errors in both the optical map data and the conventional sequence data. Despite these concerns, the alignments depicted in FIG. 4 show a high degree of correspondence and serve to flag errors in both sequence assembly and map construction.

Several discrepancies between the optical maps and the conventionally-generated sequence data were detected upon alignment. Notably, the absence of a 450 kb region is immediately evident in the NheI optical map, which was confirmed in both the XhoI optical map and the conventional sequence data. These data showed that the preliminary NheI optical map contained an assembly error, which omitted this 450 kb region. A gap in sequence (approx. 54 kb) was also revealed when the composite optical maps were compared to sequence (“gap 2,” Perna et al, 2001, supra). Because this gap was closed after sequencing new templates derived from fractionated genomic DNA, it is not illustrated here.

Additionally, there are two small regions (approx. 6 kb and 7 kb) present in the XhoI optical map that are missing from the conventional sequence data (noted in Table 1, FIG. 2B, and FIG. 4 as “O” and “R”). These two regions could not be verified as missing using the NheI optical map, because they were within the 450 kb region that was absent from the NheI optical map. However, these regions in the XhoI optical map each had significant coverage underlying the consensus map (roughly 20 molecules). This discrepancy between the XhoI optical map and the conventional sequence data may be due to the fact that these regions coincide with phage elements that were difficult to assemble correctly because some sequence reads match the assembly in several different places where related phage are integrated.

There are also four regions where the number of fragments from the conventional sequence data do not exactly match those from the optical map. These regions are denoted in Table 1, FIG. 2B, and FIG. 4 as “1,” “2,” “3,” and “V.” Optical map data in these regions showed the absence of 1 or 2 restriction enzyme sites. “V” is another instance of partially completed sequence assembly due to the difficulty of matching sequence reads to the correct phage locus. However, when compared with a recently release E. coli sequence (generated by conventional means, Hayashi et al., 2001, DNA Res. 8:11-22, 47-52), regions 1, 2, and V of the present optical maps matched. This match, while quite promising, is not conclusive evidence that the optical map data is correct because the Hayashi et al. sequence used a different bacterial strain (RIMD 0509952) with the same O157:H7 serotype.

Construction and Analysis of Optical Maps:

Various aspects of the optical mapping and analysis techniques utilized herein are described U.S. Pat. Nos. 6,294,136; 6,221,592; 6,150,089; 5,720,928; 5,599,664; and 5,405,519. The entire contents of each of the foregoing U.S. patents is incorporated herein by reference in its entirety.

1. Surface Preparation: Unlike instances in the past in which nucleic acid molecules were attached to solid surfaces, the controlled, reproducible solid surface elongation/fixation techniques described herein utilize surfaces, especially glass surfaces, that reproducibly elongate and fix single nucleic acid molecules. As discussed in greater detail below, the surfaces described herein generally exhibit a positive charge density. Several parameters must be taken into account, however, in order to optimize the solid surface charge density such that, for example, the genome analysis techniques described herein, such as the assembly of an optical contig map, can be performed.

The solid surfaces of the invention should exhibit a positive charge density which achieves an optimal balance between several parameters, including elongation, relaxation, stability and biological activity.

The solid surface must allow the molecule to be as completely elongated as possible, while allowing for a small degree of relaxation. As used herein, “small degree of relaxation” refers to a level of relaxation which yields a gap of between about 0.5 microns and about 5.0 microns when an elongated nucleic acid molecule is cut. An optimal balance between these two parameters yields improved imaging capability. For example, an efficient balance between elongation and relaxation capability facilitates the imaging of newly-formed, growing gaps that develop at restriction enzyme cleavage sites.

In addition to elongation and relaxation, the biological activity retained by the elongated nucleic acid molecule must be taken into account when optimizing the positive charge density of the elongation/fixation solid surface. Further, the stability of the elongated nucleic acid molecules on the surface must be considered. In the case of a restriction digest (that is, as part of an optical mapping procedure), “stability” refers to how well the restriction fragments formed are retained on the solid surface.

As a first step toward determining the positive charge density which represents an optimal balance between each of these parameters, the positive charge density (for example, the level of surface derivatization) may be titrated against the measured average molecular length of the nucleic acid molecules which are deposited on the surface. Molecule counts (i.e., the number of countable molecules which have been deposited) on the surface can also be measured.

At low levels of positive charge density, the average molecular extension on the surface is low. This may be due to the fact that, at low positive charge concentration, not enough nucleic acid binding sites exist to hold an extended molecule with stability. As the positive charge density increases, the average nucleic acid molecular extension also increases, eventually peaking. As the positive charge density continues to increase further, the average amount of molecular extension then begins to decrease. This may be due to the presence of such an abundance of nucleic acid binding sites that any flow forces that are present, and that would drive elongation of the nucleic acid molecules, are overwhelmed. As a result, molecular extension is, at least to some extent, quenched.

Once a positive charge density (e.g., the derivatization level) is achieved which affords maximum nucleic acid molecule extension, the elongation parameters must be tested within the context of the specific imaging or analysis procedure for which the single molecules are to be used. In the present instance, the molecules will be subjected to cleavage with restriction endonucleases, followed by optical mapping. Such testing involves an evaluation of the biological activity of the nucleic acid molecule, as well as a determination of the relaxation level of the elongation nucleic acid. For example, in instances wherein the elongated nucleic acid molecules are to be used for optical restriction mapping, the level of elongation/fixation must allow for cutting by the restriction enzyme. The level of elongation and fixation must also provide a level of relaxation which makes possible the ready imaging of nascent restriction enzyme cleavage sites.

In the case of optical mapping, one such test would include the digestion of a plurality of elongated nucleic acid molecules, followed by a determination of the cutting efficiency of the enzyme and a measurement of the size of the nascent gaps formed at the new cleavage sites (thus measuring relaxation). A cutting efficiency of at least about 50% is an acceptable level of biological activity retention. Acceptable relaxation levels are as described above.

Further, the stability of the elongated nucleic acid molecule must be ascertained. As discussed above, in the case of optical mapping, stability refers to the retention level of newly formed restriction fragments on the surface. For optical mapping, an acceptable stability level is one in which at least about 80% of the newly-formed restriction fragments are retained.

Solid surfaces may be prepared for optimal elongation and fixation of single nucleic acid molecules via a variety of simple manipulations. First, for example, the surfaces may be derivatized to yield a positive charge density, which can be optimized as described above. Preferably, the charge density should be proportional to the amount of derivatization. Second, simple manipulations may be performed to modulate reversibly the surface positive charge density to optimize surface charge density at each step of the nucleic acid elongation, fixation, analysis, and/or manipulation steps. Such reversible charge density modulation is referred to herein as “facultative fixation,” as discussed below. Third, additional methods for further affecting the elongation/fixation of the single nucleic acid molecules are discussed. These include, for example, methods for controlled drying, for the generation of gradients of positive charge density, and for cross-linking of the elongated nucleic acid molecules.

Surfaces may be derivatized using any procedure that creates a positive charge density that favors an interaction with a nucleic acid molecule. Any compound which absorbs to or covalently binds to the surface of interest and introduces a positive charge density onto the surface can be utilized as a derivatizing agent. Such compounds should not fluoresce.

For example, surfaces may be derivatized with amino-containing compounds that absorb to or covalently bind to the surface of interest. Such amino-containing compounds can, for example, include amino-containing silane compounds, which are capable of covalently binding to surfaces such as glass. Among these amino-containing silane compounds are 3-aminopropyltriethoxysilane (APTES) and 3-methylaminosilane. APTES is quite useful in that it may be cross-linked. The use of 3-methylaminosilane may, in certain instance, be of greater advantage in that the compound resists oxidation.

Derivatizing agents that non-covalently absorb to surfaces, such as glass surfaces may, include, for example, poly-D-lysine (polylysine). Polylysine binds to glass via electrostatic interactions. When utilizing polylysine as a derivatizing agent, the size of the polymeric polylysine is to be taken into account. For example, low-molecular weight polylysine (e.g., less than 200,000 Da; with about 90,000 Da being preferred) appears to fix elongated nucleic acids more tightly than high-molecular weight polylysine (e.g., molecular weight greater than 200,000 Da, with greater than 500,000 Da being preferred). Thus, when elongating and fixating nucleic acids on a solid surface derivatized with polylysine, a low-molecular weight polylysine would be preferred for tighter fixation, e.g., for fixing smaller nucleic acid fragments.

Surface derivatization may be achieved by utilizing simple, reproducible techniques. When derivatizing a surface with APTES, for example, a clean surface, such as a glass surface, may be incubated in an acidic APTES solution for a given period of time. Increasing the incubation time increases the resulting charge density of the surface. It is preferred that conditions should be chosen such that the single nucleic acid molecules are elongated to approximately 50-100% of their polymer contour length.

In one embodiment of such an APTES derivatization procedure, a clean glass surface can be incubated for an appropriate period of time in an APTES concentration of about 0.10 M, pH 3.5, at a temperature of about 65° C. Incubation times for such an embodiment can range from about 3 hours to about 18 hours. To stop the derivatization process, the surfaces need only be removed from the APTES solution and repeatedly rinsed in high-purity water. Clean, derivatized surfaces are then air dried.

With respect to derivatizing a surface with polylysine, a clean surface, such as a glass surface, can be derivatized in a polylysine solution. The concentration and molecular weight of the polylysine used for derivatization affect the level of derivatization achieved per incubation time. Increasing the polylysine concentration increases the resulting surface charge density which forms. For optical mapping purposes, conditions should be chosen such that single nucleic acid molecules are extended up to about 100% of their polymer contour length.

In one embodiment of such a polylysine derivatization method, a clean glass surface can be incubated overnight, at room temperature, in a solution of polylysine having a molecular weight of about 350,000 Da, at a concentration of about 10⁻⁶ to 10⁻⁷ grams per milliliter. After incubation, the derivatized glass surface is rinsed in highly pure water and either air dried or wiped dry with lens tissue paper. Such conditions are expected to achieve nucleic acid elongation levels which are suitable for optical restriction mapping.

In addition to methods which involve the use of a derivatizing agent such as described above, a positive charge density may be introduced onto a surface by a number of alternate means. Such a positive charge density may, for example successfully be applied to a surface via plasma derivatization, an electrostatic generator (to create electrical charge), or corona discharge. In short, the manner in which the positive charge density is generated is not overly critical to the ultimate success of the invention, so long as the method chosen yields a surface having the appropriate positive charge density such that nucleic acid molecules can be fixed on the surface with the required amount of elongation and relaxation.

Described herein are methods for the reversible modulation of solid surface positive charge density. Such methods are designed to optimize solid surface charge density at each step of the elongation, fixation, and analysis/manipulation steps described herein. Among the ways by which such a reversible charge density can be effected include changes in the salt concentration, divalent cation concentration, effective water concentration, and/or pH. This approach to optimizing surface charge density during the entire optical mapping process is referred to herein as “faculative fixation.”

Using facultative fixation, the surface positive charge density can be tailored to suit each step of the single nucleic acid techniques described herein. For example, it may be desirable to fix the nucleic acid molecule under reversible conditions which favor a loose charge density, leading to a higher degree of nucleic acid molecule spreading. The charge density may then, for example, be increased for a restriction digest step. Additionally, it may be desirable to digest a molecule so tightly fixed that no relaxation gaps form upon cleavage and then to subsequently lower the charge density such that the gaps are allowed to form. Finally, a very high charge density may then be chosen if the sample is to be stored. That is, the newly formed restriction fragments are firmly adhered to the surface so that they do not detach from the surface during storage.

With respect to salt concentration, as the salt concentration at the surface/solution interface increases (e.g., from 0 to 5 M NaCl), the surface positive charge density decreases. With respect to divalent cation (e.g., Mg²⁺, Ca²⁺) concentration, as the divalent cation concentration at the surface/solution interface increases (e.g., 1 mM to 1 M), the surface positive charge density decreases. As the effective water concentration is decreased, due to the addition of an increasing concentration of non-aqueous material, the surface positive charge density increases.

Changing the pH represents a gentle and fast method to modulate the charge density of a surface reversibly. A low pH promotes a positively-charged environment, while a high pH promotes a less-positively charged, more neutral environment.

Taking, as an example, a surface which has been derivatized using an aminosilane compound, a pH of approximately 6 yields a positive charge density. Raising the pH lowers the charge density until the charge is essentially neutral at a pH of 9-10. A variety of simple methods may be utilized to produce pH-based facultative fixation. For example, the surface can be exposed to buffers, such as Tris or phosphate buffers, of varying pH. Additionally, gas-induced pH changes can be made. For example, CO₂ gas can be introduced over the buffer in which the derivatized surface is submerged such that the buffer is acidified, thereby increasing the overall charge density on the surface. Alternatively ammonia gas, for example, may be introduced over the buffer, raising the buffer pH, thereby lowering the overall surface charge density. These latter gas-based techniques are especially useful in instances wherein it is essential to minimize possible physical disturbances on the solid surface in that the buffer remains undisturbed throughout the facultative fixation process.

Other methods of inducing positive charge density on the surface include, for example, derivatization gradients. In addition to a uniform, controllable derivatization of an entire solid surface, it is also possible to form a gradient of derivatization. Such a derivatization gradient can be formed by, for example, the use of drops of derivatizing agents deposited on the solid surface. Upon deposition, such a drop would form a meniscus, leading to a greater concentration of derivatizing agent available to the solid surface at the perimeter of the drop than within its interior section. This, in turn, leads to a gradient of derivatization, with the outer perimeter of the treated surface exhibiting a higher level of derivatization than the interior.

Such a gradient of derivatization promotes a higher percentage of fully elongated molecules. Further, due to the tension set up across the nucleic acid molecule, a more efficient level of aligning and packing is observed, thus maximizing the amount of usable molecules per imaging field. Increasing the number of useful molecules per imaging field is greatly desired because it speeds the overall process of assembling an optical map.

Cross-linking may also be used to induce positive charge density on the surface. The elongated nucleic acid molecules may be cross-linked to the solid surface. Such cross-linking serves to fix the molecules to the surface permanently. This can be advantageous for a variety of reasons. For example, cross-linking may be useful when working with very large nucleic acid molecules. Further, the surface properties of the solid may be modulated with no possibility of nucleic acid loss. Additionally, the possibility of unacceptable nucleic acid fragment loss or relaxation which could occur over the course of, for example, storage or a long reaction, is eliminated upon cross-linking.

Cross-linking, as utilized herein, is to be performed in conjunction with the elongation/fixation techniques described herein. First, the desired level of elongation is determined and achieved, and subsequent to this, the elongated nucleic acid is cross-linked for permanent fixation. A number of cross-linking methods are available, including glutaraldehyde and UV cross-linking. Glutaraldehyde cross-linking may be performed using, for example, a 5-minute incubation period in a 10 mM glutaraldehyde solution. UV cross-linking may be accomplished using, for example, a Stratalinker (Stratagene, La Jolla, Calif.) cross-linker, following the manufacturer's protocols.

Controlled drying may also be used to control the charge density of the surface. Additional compounds may be added to the aqueous solution by which the nucleic acids may be deposited onto the solid surfaces (see below for deposition techniques). These additional compounds yield drying characteristics that promote the production of a greater percentage of fully elongated nucleic acid molecules and which exhibit a lower level of intermolecular overlap or tangling, both features of which are extremely useful for analysis purposes.

Compounds which may be added for such a controlled drying aspect of the elongation methods include, but are not limited to glycerol, DMSO, alcohols, sucrose, neutral polymers such as Ficoll, and dextran sulfate. While their mechanisms of action are not known, it is possible that these compounds promote a liquid crystalline state which promotes the above-described features.

Hydrophobic regions may also be introduced onto portions of the solid surfaces. These hydrophobic regeions can serve as “microwells.” These hydrophobic regions create closed boundaries, which make it possible to introduce different reagents onto different portions of the solid surface. In this fashion, a number of different reactions can be performed simultaneously on the same solid surface.

The solid surfaces of the invention may also be prefixed with agents of interest prior to the introduction of the nucleic acid molecules to be elongated. For example, proteins may be fixed onto the solid surfaces by routine means, such as cross-linking means, which are well known to the skilled artisan. Among the proteins that can be prefixed onto the solid surfaces of the invention are enzymes, such as restriction enzymes, which are used to manipulate nucleic acid molecules, or any other nucleic acid-binding proteins. Thus, upon elongation of nucleic acid molecules onto the solid surfaces containing such prefixed enzymes, and the addition of whatever additional agents (such as certain divalent ions) that are necessary for the enzymes to act upon nucleic acids, the single nucleic acid molecules can be manipulated. In the case of restriction endonucleases, for example, using the prefixation technique yields fixed/elongated nucleic acid that is cleaved at appropriate restriction sites. Using the prefixation technique also enable a number of different reactions to be performed simultaneously on the same surface.

2. Nucleic Acid Molecule Deposition: As described above, a wide size range of nucleic acid molecules may be deposited onto the derivatized solid surfaces described herein. Specifically, nucleic acid molecules from about 300 base pairs to greater than 1000 kb can be analyzed using such solid surfaces. Smaller nucleic acid molecules, which are relatively shear resistant, can be isolated using standard nucleic acid purification techniques well known to those of skill in the art. These smaller nucleic acid molecules may be less than about 150 kb and, generally, are less than about 20 kb.

Larger nucleic acid molecules, which are subject to breakage by shearing events, can be isolated by utilizing nucleic acid molecule isolation techniques known in the art. Such shear-sensitive nucleic acid molecules are generally greater than 150 kb, but may include molecules greater than about 20 kb.

Such methods for large nucleic acid molecule isolation include, for example, agarose-embedded cell lysate techniques as described in U.S. Pat. No. 4,695,548 (incorporated herein by reference). Briefly, cells are washed, mixed with molten low-melt agarose, which is then allowed to set. The resulting block is placed in a lysis solution containing EDTA, protease, and detergent which diffuses into the block, lysing the cells and rendering intact naked DNA molecules stripped of their associated proteins. The absence of physical manipulation keeps the DNA essentially intact. The agarose can then melted and the DNA can be subjected to elongation and fixation techniques. Alternatively, chromosomal DNA can first be resolved into chromosomal populations via standard methods such as, for example, pulsed-field gel electrophoresis (PFGE).

Additionally, a condensation agent is used to collapse gel-bound nucleic acid molecules into small, shear-resistant balls, that can be unfolded with the addition of an ionic compound, such as sodium chloride or magnesium chloride. Preferably, the condensation agent is spermine as described in U.S. Pat. No. 5,720,928 (incorporated herein by reference). While spermine is preferred, other suitable materials for collapsing such nucleic acid molecules include any material or condensation agent which can cause a particular nucleic acid molecule to collapse, e.g., any condensation agent which causes nucleic acid molecules to solvate themselves preferentially. Additional examples of such materials include, but are not limited to, spermidine, alcohol and hexamine cobalt.

Larger nucleic acid molecules (i.e., those greater than about 90 kb) should, generally, be deposited onto the solid surfaces in a manner which minimizes breakage due to shear forces. Preferably, therefore, the nucleic acid molecules deposited in such an aqueous fashion can be elongated by simply allowing the aqueous solution to dry. Thus, in the absence of any manipulations, apart from simple deposition onto a derivatized surface made according to the present invention, single nucleic acid molecules can efficiently, successfully, and rapidly generate elongated and fixed nucleic acid molecules suitable for imaging and/or further manipulation. Such a technique is especially well-suited to high throughput analysis techniques.

As described previously, elongated and fixed DNA molecules (from roughly 2 to 1,500 kb) can be obtained using the flow and adhesion forces generated when a fluid sample is compressed between two glass surfaces, one of which is derivatized with polylysine or APTES (see U.S. Pat. No. 5,720,928, incorporated herein by reference). Molecules thus fixed can be digested with restriction endonucleases, fluorescently stained with, for example, YOYO-1 (oxazole yellow dimer), and optically mapped (Cai et al., 1995, Proc. Natl. Acad. Sci. USA 92:5164-5168). To increase the throughput and versatility of optical mapping, multiple samples need to be arrayed on a single mapping surface. Although robotic gridding techniques for DNA samples exist (Heller et al., 1997, Proc. Natl. Acad. Sci. USA 94:2150-2155; Craig et al., 1990, Nucleic Acids Res. 18:2653-2660; and Nizetic et al., 1991, Proc. Natl. Acad. Sci. USA 88:3233-3237), such approaches were not designed to work with single molecule substrates and could not be relied upon to deposit molecules retaining significant accessibility to enzymatic action.

To examine molecular effects that would ensure a usable population of elongated molecules, several new approaches to molecular deposition were investigated, based on placing small droplets of DNA solution onto critically derivatized glass surfaces. A new macromolecular effect which readily elongates and fixes DNA molecules was discovered, characterized, and is referred to herein as “fluid fixation.”

Fluid fixation uses the flows developed within a drying droplet (via evaporation) to elongate and fix DNA molecules to charged surfaces. Conveniently, application of outside forces are completely obviated, making the use of electrical fields, a traveling meniscus, (Michalet et al., 1997, Science 277:1518), or end-tethering of molecules with beads (Strick et al., 1996, Science 271:1835-1837) unnecessary. The passive nature of fluid fixation provides the platform needed to automate optical mapping. In addition, the biochemical versatility of fluid-fixed molecules is demonstrated by the imaging of DNA polymerase I action on these substrates.

Given the ability to grid multiple samples, and to assay biochemistries on a single-molecule level, an integrated system has been developed to deposit samples robotically, and to image substrate molecules using automated fluorescence microscopy. In general, fluid fixation of nucleic acid molecules is performed by spotting droplets of liquid containing the nucleic acid molecules onto derivatized surfaces and allowing the droplets to dry.

In a preferred embodiment, double-stranded nucleic acid molecules are elongated, aligned and fixed by spotting droplets of DNA solution onto derivatized glass surfaces using a glass capillary tube (500 μm, i.d.) or a truncated stainless steel syringe needle. The capillary or needle is used to draw DNA samples into the tube by capillary action and then to spot the DNA onto the derivatized glass surfaces by simple contact. In one embodiment, the droplets were 10 to 20 nL and contained 5-50 ng/μl of DNA in Tris-EDTA buffer). The capillary tube or needle is operated using an Eppendorf micro-manipulator in combination with an x-y table (interfaced with a computer) controlled by a microstepper motor. Preferably, the spots are 500 to 1000 μm in diameter. More preferably, the spots are 500 to 900 μm, and most preferably 900 μm+100 μm. The samples are allowed to air dry.

In a more preferred embodiment, addition of either glycerol or other polyalcohol “dopants” to the spotting solutions maximizes the elongation and alignment of the nucleic acid molecules and minimizes overlapping.

3. Enzymes for Use in Manipulating Nucleic Acids: The methods of imaging a labeled nucleotide may utilize enzymes for nicking the individual double-stranded nucleic acid molecules, opening the nicked sites, and for adding labeled nucleotides.

In one embodiment of the invention, the nicking step of the method for imaging the addition of a single labeled nucleotide is performed by the enzyme DNase I. E. coli DNase I nicks DNA in the presence of Mg⁺², an activity easily modulated by DNase concentration or time. The level of DNase I action must be controlled so as to obtain nick sites that are spaced far enough apart on average to minimize optically coincident addition sites. This enables the imaging of discrete, non-coincident sites. One skilled in the art is able to use known experimental methods to maximize the number of addition sites on a molecule for high throughput.

Assays for DNase I activity can be used by one of skill in the art to optimize the amount of nicking of the surface-fixed double-stranded nucleic acid molecules. For example, the following variables can be systematically adjusted and the results compiled: the concentration of the enzyme, the time of incubation, the buffer composition, and the surface conditions. Histograms from these various optimization runs can be analyzing using the machine vision/analysis system as described herein, thereby accumulating large numbers (1,000 to 10,000) of molecule samples indicating how different conditions affect the nick efficiency. From such analysis, one can determine the optimum conditions. Since nick translation activity is sequence-context dependent, conditions should be selected to minimize such sequence-context dependent activity.

In another preferred embodiment, the nicked site of the double stranded nucleic acid molecule is opened using T7 exonuclease gene 6. T7 exonuclease gene 6 acts by a distributive mechanism at nick sites and double-stranded ends. This enzyme is used to open nicked sites to generate gapped duplexes as substrates for Sequenase and for Klenow polymerases, and is used to create gaps of about 20 to 40 nucleotides. The formation of excessively large gaps could lead to double-strand breaks, especially if nick sites on opposite strands are near each other.

Gapping activity is assayed by treating surface-mounted molecules with DNase I followed by T7 exonuclease and then tabulating the cut sites. One skilled in the art knows to use optimized DNase concentration before treating with T7 exonuclease.

One skilled in the art would be able to optimize conditions for using T7 exonuclease gene 6 to obtain optimal nicking for optical sequencing. By way of example, parallel experiments are run to estimate gap size and the incidence of double-stranded breaks. To estimate the average gap sizes, T7 exonuclease reactions are run using lambda DNA or cosmid DNA in varying conditions, then incorporating radiolabeled nucleotides with Sequenase, followed by denaturing gel electrophoresis (generating fragment sizes amenable to standard sequence gels). A “spectrum” of additions is observed. Further, a phosphor imager can be used to measure yields. In a parallel experiment, agarose gels are run to determine the extent of double-stranded breaks.

In another embodiment, addition of a single or multiple labeled nucleotides is performed by a polymerase.

In preferred embodiments of the present invention, the polymerase is DNA Polymerase I, the Kienow fragment of DNA Polymerase I lacking the 5′-3′ exonuclease activity, T7 Sequenase v. 2.0, or Taq polymerase. Additionally, 5′-3′ exonuclease activity can be suppressed by the addition of nucleotide monophosphates.

DNA Polymerase I has been used in nick translation reactions of DNA molecules deposited onto optical mapping surfaces (New England Biolabs, Beverly, Mass.). Polymerase I vigorously incorporates pure, fluorochrome-labeled nucleotides (no unlabeled nucleotides are required for addition). The 5′-3′ exonuclease activity of the enzyme provides a convenient route for simple incorporation of labeled nucleotides at nick sites and obviates the need for gap formation on native target DNA.

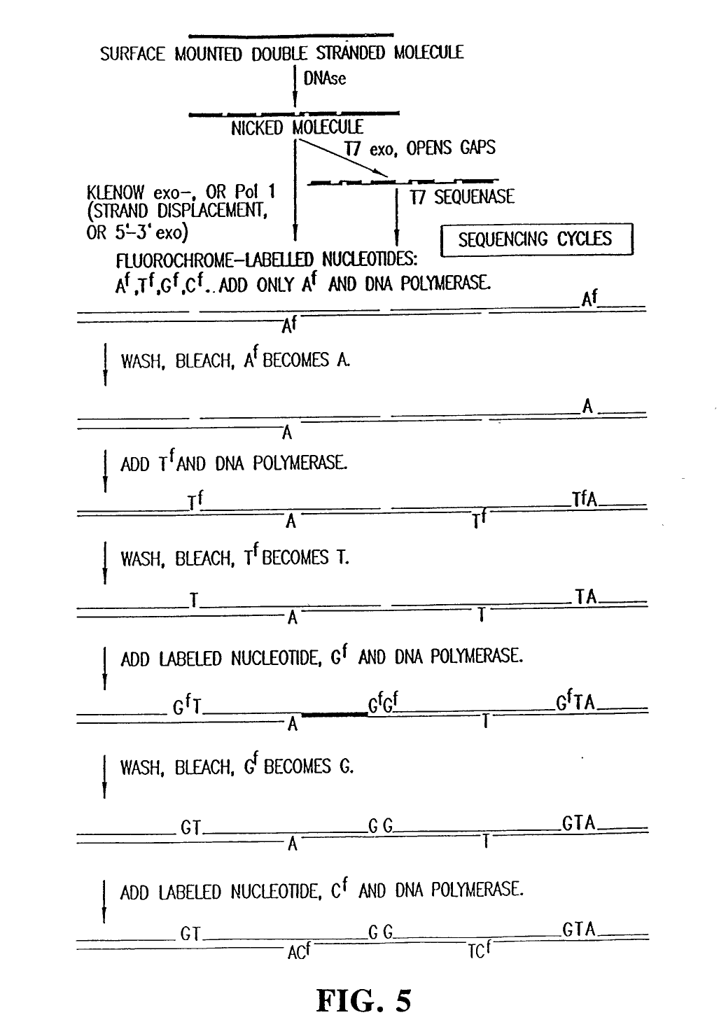

However, the 3′-5′ proof-reading ability may cause problems. When a single nucleotide is added in the presence of DNA polymerase I, there is the opportunity for exonuclease activity to remove nucleotides or “chew back” beyond the nascent addition site, obviously destroying any chance for sequence determination. This activity is suppressed when a nucleotide matching the template strand is included. However, at any given time in the optical sequencing cycle, there can be up to three other non-matching, and thus vulnerable bases exposed in template strands (see FIG. 5, describing the chemistry of optical sequencing). Addition of all four nucleotides would confound this method for optical sequencing.

There are several strategies for suppressing the 3′-5′ exonuclease activity known to those of skill in the art, such as: high nucleoside monophosphate concentration to compete against the nascent strand for the 3′-5′ exonuclease binding site, maintaining a low temperature to minimize frayed ends (16° C., or perhaps below; balancing enzyme activity), or using an exo-mutant. Another approach is to use primer extension reactions instead of nick translation.

In a more preferred embodiment, the commercially-available Klenow fragment with ablated proofreading activity can used in the present invention (Bebenek et al., 1990, J. Biol. Chem. 265:13878-13887). The reason to use primer extension is that all templates are the same. Nucleoside monophosphate does suppress proofreading, but it is not sufficiently reliable for optical sequencing.

Another embodiment of the present invention uses the Klenow fragment of DNA Polymerase I which is commercially available as a 3′-5′ exonuclease(−) mutant (Amersham, Piscataway, N.J.). Compared to polymerase I, the lack of proofreading is a distinct advantage for the reasons described above. However, lack of 5′-3′ exonuclease activity can cause problems of template switching during strand displacement or diminished activity on adsorbed molecules. Lack of proofreading also affects addition fidelity, although this problem can be minimized by limiting the number of additions to, perhaps, no more than 20 nucleotides.

Klenow activity on surface-mounted nucleic acid molecules can be assayed using methods commonly known to those skilled in the art. By means of example and not limitation, Klenow nucleotide incorporation activity can be measured by generating nicks in the surface-mounted double-stranded nucleic acid molecules using T7 exonuclease gene 6 (as discussed above) and then adding either mixtures of fluorochrome-labeled and unlabeled nucleotides or only labeled nucleotides. The rates of fluorochrome incorporation (in terms of sites and amounts) is determined by constructing histograms of images containing 1,000 to 10,000 molecule substrates as functions of time, temperature, surface variables, and buffer conditions.

Primer extension assays known to those skilled in the art can also be utilized to determine the ability of Klenow or DNA Polymerase I to act on surface-mounted molecules within a sterically-confined environment. For example, by changing buffer pH or salt concentration (within a range of enzyme functionality), electrostatic forces responsible for molecular adhesion to the surface can be altered. The protonization of the amine groups on the surface reduces effective charge, and increasing salt concentration reduces effective charge on both surface-bound amines and DNA molecules.

Another preferred embodiment of the present inventions utilizes the polymerase, T7 Sequenase v. 2.0 (Amersham) which lacks a 5′-3′ or 3′-5′ exonuclease activity, but, unlike Polymerase I, its action is processive. Also, this enzyme does not exhibit strand displacement activity.

In a preferred embodiment, the T7 exonuclease gene product 6 (from Amersham) is used to create small gapped duplexes at nick sites which is followed by use of the T7 Sequenase v. 2.0 for incorporation of labeled nucleotides.

Where sequence-specific, double-stranded nucleic acid cleavage is desired, as when generating optical contig maps, any restriction endonuclease (i.e., restriction enzymes) now known or discovered in the future can be used. Whole-genome optical contig maps generated the enzymes NheI and XhoI are described in the Examples. Other suitable restriction enzymes that can be used in the present invention include, without limitation: Aat II, AccI, AccIll, Acc65 I, AccB7 I, Acy I, Age l, Alu l, A/w26 I, A/w441, Apa l, Ava I, Ava 11, Ba/I, BamH l, Ban I, Ban II, Bbu l, Bc/I, Bgl l, Bg/Il, BsaM I, BsaO I, Bsp1286 I, BsrBR I, BsrS I, BssH II, Bst71I, Bst98 I, Bst E II, Bst O I, Bst X I, Bst Z I, Bsu36 I, Cfo I, Cla l, Csp I, Csp 45 I, Dde I, Dpn I, Dra l, EclHK I, Eco47 III, Eco52 I, Eco72 I, EcoI CR I, EcoR I, EcoR II EcoR V, Fok I, Hae ll, HaelIl, Hha I, Hinc II, Hind III, Hinf I, Hpa I, Hpa II, Hsp92 I, Hsp92 II, I-Ppo I, Kpn I, Mbo I, Mbo II, Mlu l, Msp I, MspA I, Nae l, Nar, Nci I, Nco I, Nde l. NgoM I, Nhe I, Not I, Nru I, Nsi l, Pst l, Pvu l, Rvu II, Rsa l, Sac I, Sac II, Sal l, Sau3A I, Sau96 I, Sca l, Sfi I, Sgf I, Sin I, Sma l, SnaB I, Spe l, Sph I, Ssp l, Sst I, Stu l, Sty l, Taq I, Tru9 I, Tthlll I, Vsp I, Xba I, Xho I, Xho II, Xma l, and Xmn I. All of the enzymes in this list are commercially available from a number of sources, such as NEB and Promega Corporation (Madison, Wis.). In the same fashion as when nicking double-stranded DNA, one of skill in the art is able to optimize reaction conditions to afford sequence-specific cleavage of an elongated and fixed nucleic acid molecule using any of the above-noted restriction enzymes.

4. Labeled Nucleotides: Numerous labeled nucleotide molecules are commercially available for use in the present invention. In a preferred embodiment of the invention fluorescently-labeled nucleotides are used. By way of example, and not limitation, the present invention uses nucleotides labeled with fluorescein, rhodamine, cyanine, or pyrene. These fluorochromes, as well as a host of others are available commercially from Molecular Probes, Inc., Eugene, Oreg. Particularly suitable and commercially-available fluorochromes from Molecular Probes include 6-(((4-(4,4-difluoro-5-(2-thienyl)-4-bora-3a,4a-diaza-s-indacene-3-yl)phenoxy)acetyl) amino)hexanoic acid, succinimidyl ester (“BODIPY TR”), 6-((4,4-difluoro-1,3-dimethyl-5-(4-methoxyphenyl)-4-bora-3a,4a-diaza-s-indacene-2-propionyl) amino) hexanoic acid, succinimidyl ester (BODIPY TMR), and cyanine-based dyes such as “YOYO”-brand dyes.

In a more preferred embodiment, Perkin Elmer (“PE”) Applied Biosystems (Foster, Calif.) sells fluorescent dNTPs that have been used successfully in nick translation experiments for several years. PE offers two nucleotides, dUTP and dCTP, each conjugated with three different rhodamine fluorochromes, R110, R6G, and TAMRA. These nucleotide derivatives were originally developed for incorporation at high yields in PCR reactions,-to be analyzed by automated gel electrophoresis. In many ways, their use in the present invention is less demanding than PCR amplification because the template strands remain the same throughout the optical sequencing reaction cycles.

The chemical and optical features of these nucleotides make them ideal for optical sequencing: high incorporation yields by different polymerases (Taq DNA polymerase or other thermostable DNA polymerases, DNA polymerase I {Perkin-Elmer Applied Biosystems, [F] dNTP Reagents, Protocol 402774, 1996}, or Sequenase {Amershan}), good fluorescence yields, and the availability of three different fluorochromes for conjugation, providing a route for multiplexing.

The fluorochrome should ideally: (1) conjugate to nucleotides but not hinder the action of polymerase enzymatic action and activity; (2) emit sufficient numbers of photons to provide an image; and (3) be capable of photobleaching.

5. Imaging: The single- or multiple-labeled nucleotides added to the individual double-stranded nucleic acid molecules of the present invention can be imaged via a number of techniques to generate a digital image of the label or cleavage site which can then be processed to obtain quantitative and qualitative measurements of molecular parameters of interest. For example, single fluorochromes can be observed using video rate imaging techniques known to those skilled in the art (see Schmidt et al. 1996, Proc. Natl. Acad. Sci. USA 93: 2926-2929).

In one embodiment, the individual nucleic acid molecules containing the labeled nucleotides are imaged through a fluorescent microscope with a camera and illuminated with a light source. In a particular embodiment, the standard fluorescent microscope is a Zeiss Axiovert 135, x100 Plan neofluar objective. In other embodiments, the camera is a cooled CCD camera or an Intensified Silicon Target (ISIT) cooled CCD camera. Additionally, a silicon-intensified target (SIT) camera is used for focusing.

Additionally, the nucleic acid molecules mounted on a surface are covered with 45% β-mercaptoethanol with 1 mM YOYO-3 when R 110-dUTP is used and 20-30% β-mercaptoethanol with 1 mM YOYO-1 in Tris-EDTA buffer when R6G-dUTP is used as an anti-photobleaching reagent to improve the fluorochrome photobleaching half-lives by as much as 500-fold.

The elongated and fixed nucleic acid molecules with labeled nucleotides can be illuminated with an appropriate light source known in the art. By way of example and not limitation, the light source is a laser. More particularly, the laser is an Ar⁺ laser.

Further, an additional aspect of the invention entails imaging the individual nucleic acid molecules in order to map the locations of the added labeled nucleotides or cleavage events within individual nucleic acid molecules.

The elongated, fixed single nucleic acid molecules can also be imaged via a number of techniques to generate a digital image of the molecule which can be processed to obtain quantitative and qualitative measurements of molecular parameters of interest. To this end, in a preferred embodiment of the present invention, the molecules being imaged are stained with fluorochromes which are absorbed by the molecules generally in proportion to their size. Accordingly, the size of the stained molecules can later be determined from measurements of the fluorescent intensity of the molecule which is illuminated with an appropriate light source, as known in the art. (See U.S. Pat. No. 5,720,928.)

A preferred embodiment of the present invention is first to image the incorporated fluorescently-labeled nucleotides or cleavage events and then to counter-stain the individual double-stranded nucleic acid molecules to image the molecule so as to map the sites of additions or clevages. Counter-stains such as YOYO-1 and YOYO-3 are available commerically (Molecular Probes).

6. Analysis of Digital Images: The present invention also entails methods of analyzing images of single nucleic acid molecules, enzymatically cleaved in a sequence-specific fashion, in order to correlate the cleavage events, to construct an optical, genome-wide contig map, and to compare the map with a known sequence for the same genome. The method of analysis can also used to sequence the nucleic acid or to obtain the location and identification of a single nucleotide polymorphisms of a population of individual nucleic acid molecules. Methods of analyzing images of signals from labeled or cleaved molecules and correlating them to a position known in the art are specifically encompassed by the present invention.

In a preferred embodiment, the present invention analyzes the images from enzymatically cleaved nucleic acid molecules to correlate them with known nucleic acid sequences generated by other means. Thus, the present invention provides a method for validating or refuting previously generated sequences. The present invention discloses a novel method of analysis utilizing Bayesian estimation to correlate the images of cleavage events to construct a genome-wide optical contig map.

Specifically, the method of analyzing the images using Bayesian estimation, comprises the steps of:

(a) accumulating signals of a cleavage site in the image;

(b) filtering the signals according to fluorescence intensity;

(c) correlating the signals with the backbone of the nucleic acid molecule;

(d) tabulating cleavage sites of the image using Bayesian inference estimation of the signals; and

(e) aligning and assembling the cleavage sites to yield a contig map.

The analysis first requires the accumulation of fluorescent signals from a cleavage site (or addition site) of the image, or “spot” histories, as a function of position (x,y) and addition cycle I(s). Positional data of fluorescence intensities are accumulated after each cycle and are used to link labeled nucleotide additions/cleavages for a given nick or gap site. For example, the microscope field of view has many nucleic acid molecules each containing 10 to 20 nicked or cleaved sites, and the molecules vary in the size of the target and the frequency of the nicked/cleaved sites.

Next, the signals from the fluorescently labeled nucleotides are filtered according to fluorescence intensity. The signals having insufficient or excessive fluorescence intensities are rejected as false signals. The criteria for this selection is based on an accurate measure of fluorochrome addition number. Depending on the set criteria, additions are given “scores” to measure how much they deviate, and the additions with low “scores” may be ultimately rejected in a Bayesian inference scheme.

Confidence estimates and error checking can then be applied to the raw sequence data (or cleavage data) based on the addition (cleavage) history of a given nick site. A number of failure modes can occur that cause a site to be assigned a low “score.”Examples of failure modes include: template damage can cause incomplete or spurious additions; and excessive nucleotide addition caused by opening a cryptic nick site after nuclease treatment.

After completion of the sequencing cycles (or restriction enzyme digestion), the nucleotide addition signals (cleavage sites) are then correlated with the nucleic acid molecule backbone or restriction fragments if the signals receive a sufficient confidence value, C_{b}. The assignment of confidence values: (1) aids in eliminating noise, thus only events associated with the target molecules will be considered; and (2) helps to correlate events according to position, for verification and eventual assembly of the finished sequence or map.

The Bayesian estimation algorithms developed and set forth in Anantharaman et al. (1997, J. Comp. Biol. 4:91-118) are used to create the optical restriction fragment maps of the nucleic acid molecules and to correlate the labeled nucleotides and/or nucleotide sequence to the nucleic acid molecules as described below.

7. Algorithm for Making Ordered Restriction Maps to Align Nucleotide Sequences: The focus of this section is on the description of a probabilistic approach to constructing ordered restriction maps based on the data created from the images of population of individual DNA molecules (clones) digested by restriction enzymes in order to align the nucleotide sequence of individual molecules. Specifically, disclosed in detail are map-making methods and algorithms capable of producing high-resolution, high-accuracy maps rapidly and in a scalable manner to align optically-obtained nucleotide sequences along the individual nucleic acid molecule. The resulting methodology, embodied in computer program modules, is a key component of the optical mapping automation tools in accordance with the present invention.

As discussed in the preceding sections, optical mapping is a single molecule methodology for the rapid production of ordered restriction maps from individual nucleic acid molecules. Recent technological advances have led to accurate size estimates of the restriction fragments and have been used to construct final restriction maps. Nevertheless, the accuracy of restriction maps created from single molecules is fundamentally limited by the resolution of the microscopy, the imaging system (CCD camera, quantization level, etc.), illumination, and surface conditions, and other factors. Furthermore, depending on the digestion rate and the noise inherent to the intensity distribution along the molecules being imaged, it is likely that a small fraction of the restriction sites will be missed, or that spurious sites will be introduced. Additionally, sometimes (rather infrequently) the exact orientation information, i.e., whether the left-most restriction site is the first or the last, is lacking.

As a result, it should be expected that two arbitrary single molecule restriction maps for the same DNA clone obtained this way will at most be “roughly” the same, in the sense that most of the restrictions sites will appear roughly at the same place in both maps if they are aligned (i.e., have the same orientation) and if the identified restrictions sites differ by a small amount.

There are two fundamental approaches to improving the accuracy and resolution of such maps: (1) improve the chemical and optical processes to minimize the effect of each error source; and (2) use statistical approaches where the restriction maps of a large number of identical clones are combined to create a high-accuracy restriction map. Clearly, these two approaches are not mutually exclusive and various trade-offs exist that can be exploited fruitfully. In accordance with the present invention the problem is attacked by improving all aspects of the process, including the chemical, optical, computational, and automation aspects.

Chemical improvements include the use of fixed, elongated nucleic acid molecules onto positively-charged glass surfaces as described above, which improves sizing precision, as well as throughput for a wide range of cloning vectors (cosmid, bacteriophage, and yeast or bacterial artificial chromosomes). Further improvements include, without limitation: the development of a simple and reliable procedure to mount large DNA molecules with good molecular extension and minimal breakage; the optimization of the surface derivatization; maximizing the range of usable restriction enzymes and retention of small fragments; and the development of an open surface digestion format, which facilitates access to samples and lays the foundations for automated approaches to mapping large insert clones.

A complementary set of improvements, which is the focus of this section, have come from the use of powerful statistical tools to process a preliminary collection of single-molecule restriction maps, each one created from an image of a DNA molecule belonging to a pool of identical clones. Individual restriction maps in this collection are almost identical with small variations resulting from sizing errors, partially-digested restriction sites and “false” restriction sites and can be combined easily in most cases. However, the underlying statistical problem poses many fundamental challenges. For example, as shown in the following subsection, the presence of some uncertainty in the alignment of a molecule (both orientation and/or matching in the sites) in conjunction with either false cuts or sizing error is sufficient to make the problem NP-complete, that is, computationally infeasible. Garey and Johnson, 1979, “Computer and Intractability: A Guide to the Theory of NP-Completeness,” W. H. Freeman and Co., San Francisco, Calif. See also Anantharaman et al. 1997, J. Comp. Biol. 4(2):91-118 for some related results on the complexity of this problem. It should be noted that these negative results generally correspond to pathological cases that are less likely to occur in real life. Nonetheless, these negative results play an important role in clarifying the care needed in structuring the algorithm properly. The probabilistic algorithms (using a Bayesian scheme) in accordance with the present invention can handle this problem adequately.

The remainder of this section is devoted to describing the restriction map model used in accordance with the present invention, along with a formulation of the underlying algorithmic problems. Statistical models for the problem, in accordance with a preferred embodiment of the present invention, and based on certain assumptions about the distributions of the bases in DNA and the properties of the chemical processes involved in optical mapping are also described. These models are then used to devise probabilistic algorithms with good average time complexity. The algorithms implemented in computer software in accordance with the present invention cause a computer to produce several output maps ranked by a “quality of goodness” measure. Additionally, estimates of several auxiliary parameters are given, governed by the underlying chemical, optical and image analysis processes (e.g., the digestion rate, false-cut rate, sizing error, contamination with other molecules, etc.). Lastly, in the Examples, experimental results are presented for a genome-wide contig map for an E. coli strain of the serotype O157:H7.

The discussion that follows assumes a basic working familiarity with the tenets of Bayesian analysis and combinatorial problem-solving as applied to nucleic acid sequence analysis. Relevant background material can be found, for example, in: Karp, 1993, “Mapping the Genome: Some Combinatorial Problems Arising in Molecular Biology”, in Proc. of 25th Ann. ACM Symp. on the Theory of Computing, 278-285.

Kevles and Hood, eds., 1992, “The Code of Codes,” Harvard University Press, MA. Nicholl, 1994, “An Introduction to Genetic Engineering,” Cambridge University Press. Pevzner, 1990, “DNA Physical Mapping,” in Computer Analysis of Genetic Texts, 154-158. Primrose, 1995, “Principles of Genomic Analysis: A Guide to Mapping and Sequencing DNA from Different Organisms,” Blackwell Science Ltd., Oxford. Waterman, ed. 1989, “Mathematical Methods for DNA Sequences,” CRC Press, Florida. Waterman, 1995, “An Introduction to Computational Biology: Maps, Sequences and Genomes,” Chapman Hall. Lander and Waterman, 1988, “Genomic Mapping by Fingerprinting Random Clones: A Mathematical Analysis,” in Genomics 2, 231-239. Lander, 1995, “Mapping Heredity: Using Probabilistic Models and Algorithms to Map Genes and Genomes,” Notices of the AMS 42(7), 747-753, adapted from “Calculating the Secrets of Life,” National Academy of Sciences. Lander, 1995, “Mapping Heredity: Using Probalistic Models and Algorithms to Map Genes and Genomes (Part II),” Notices of the AMS, 42(8), 854-858, adapted from “Calculating the Secrets of Life,” National Academy of Sciences. Various algorithmic and computational complexity issues are addressed in Branscomb et al., 1990, “Optimizing Restriction Fragment Fingerprinting Methods for Ordering Large Genomic Libraries,” Genomics 8, 351-366; Goldberg et al., 1995, J. Comp. Bio., 2(1), 139-152; and Krawczak, 1988, In Proc. Natl. Acad. Sciences USA, 85, 7298-7301.

In accordance with the present invention the restriction map problem can be formulated mathematically as follows. Assuming that all individual single-molecule restriction maps correspond to the same clone, and that the imaging algorithm can only provide the fragment size estimates that are scaled by some unknown scale factor, a single molecule restriction map (SMRM) is represented by a vector with ordered set of rational numbers on the open unit interval (0,1):

Dⱼ=(s₁ⱼ,s₂ⱼ,s_{M}_{ⱼ}_{,j}), 0<s₁ⱼ<s₂ⱼ< . . . <s_{M}_{ⱼ}_{,j}<1, sᵢⱼ∈Q

where Q is the set of rational numbers.

Let Dⱼ+c (a rational c ∈ [0, 1]), denote the vector:

Dⱼ+c=(s₁ⱼ+c, s₂ⱼ+c, . . . , s_{Mj,j}+c)

where −s₁ⱼ<c<1−s_{Mj,j}.

Given a rational number s ∈ (0,1), its reflection is denoted by s^{R}=1−s. Similarly, D^{R}ⱼ, denotes the vector:

Dⱼ^{R}=(s_{M}_{ⱼ}_{,j}^{R}, . . . , s₂ⱼ^{R}, s₁ⱼ^{R}).

Note that if the entries of Dⱼ are ordered and belong to the open unit interval, so do Dⱼ+c and D^{R}ⱼ, provided that c is appropriately constrained.

Thus, the mapping problem in accordance with the present invention can be described as follows: given a collection of data (SMRM vectors):

D₁, D₂, . . . , Dₘ,

a final vector H:

H=(h₁, h₂, . . . , h_{N}) has to be computed, such that H is “consistent” with each Dⱼ. Thus, H represents the correct restriction map and Dⱼ's correspond to several “corrupted versions” of H. In accordance with the present invention, the notion of “consistency” is defined using a Bayesian formulation. The formulation depends on the conditional probability that a data item Dⱼ can be present given that the correct restriction map for this particular clone is H.

As known in the art, any such consistency requirement must satisfy certain conditions, given certain side information. For instance, if no false-cuts and accurate sizing information is assumed (even if the digestion may be partial), then it must be the case that for each j, either Dⱼ⊂H or D^{R}ⱼ⊂H. In particular, if the digestion is complete (ideal case) then all the Dⱼ's are identical up to reflection and H can be simply chosen as D₁.

In spite of the complexity of the issues associated with the formulation of the model (as discussed in detail in Anantharaman et al. 1997, J. Comp. Biol. 4:91-118), it is clear that the imaging system of the present invention provides an output having a considerable level of structure that can be exploited to obtain statistically accurate ordered restriction maps efficiently. For instance, if the digestion rate in a particular case is relatively high, then by looking at the distribution of the cuts a good guess can be made about the number of cuts and then only the data set with large numbers of cuts can be used to create the final map. Other approaches to utilizing the structure of the input have used formulations in which one optimizes a cost function and provides heuristics (as the exact optimization problems are often infeasible). In one approach, the optimization problem corresponds to finding weighted cliques; and in another, the formulation corresponds to a 0 to 1 quadratic programming problem (Muthukrishnan and Parida 1996, “Towards Constructing Physical Maps by Optical Mapping: An Effective Simple Combinatorial Approach,” in Proceedings First Annual Conference on Computational Molecular Biology (RECOMB97), pp. 209-215, ACM Press). However, these heuristic approaches have only worked on limited sets of data and their effectiveness (or approximability) in large-scale practical applications remains unproven. The present invention improves over this and other prior art approaches by providing map-making methods and computer systems capable of producing high-resolution, high-accuracy maps rapidly and in a scalable manner.

Specifically, in accordance with the present invention, a probabilistic algorithm based on a Bayesian approach is used to obtain the desired high-accuracy restriction maps. The approach is to use a carefully constructed prior model of the cuts to obtain the best hypothetical model by using Bayes' formula. (See Dempster et al., 1977, J. Roy. Stat. Soc. 39:1-38; Grenander et al. 1993, J. Roy. Stat. Soc. 56:549-603). Generally, the approach requires searching over a high-dimensional hypothesis space and is complicated by the fact that the underlying distributions are multimodal. However, as shown next, and in accordance with the present invention, the search over this space can be accomplished without sacrificing efficiency. Advantageously, the proposed algorithm is flexible in the sense of enabling the operator to trade computational speed for accuracy of the final map by constraining various parameters in the implementation. The method has been implemented and extensively tested over automatically generated data with good results.

The main ingredients of the Bayesian scheme according to a preferred embodiment of the present invention are the following:

(1) A Model or Hypothesis H, of the map of restriction sites; and

(2) A Prior distribution of the data (SMRM vectors):

Pr[Dⱼ|H]

Assuming pair-wise conditional independence of the data (SMRM) vectors Dⱼ, i.e.,

Pr[Dⱼ|Dⱼₗ, . . . ,Dⱼₘ,H]

the conditional probability of the entire data set of SMRM vectors given a hypothesis H becomes:

$$Pr [ D \vert H ] = {\prod }_{j}^{m} Pr [ {D}_{j} \vert H ] ,$$

where the index j ranges over the data set.

As is known in the art, the posterior distributions via Bayes' rule are then given by the expression:

$$Pr [ H \vert D ] = \frac{Pr [ D \vert H ] Pr [ H ]}{Pr [ D ]} \qquad ( 1 )$$

where Pr[H] is the prior unconditional distribution of hypothesis H, and Pr[D] is the unconditional distribution of the data. In accordance with a preferred embodiment of the present invention, using this formulation, the space of all hypotheses is searched to find the most “plausible” hypothesis H* that maximizes the posterior probability. This hypothesis provides the final output map in a preferred embodiment.

To compute the hypothesis H* in equation (1), one needs to compute or model the quantities on the right. In a preferred embodiment of the present invention, the hypotheses H is modeled by a small number of parameters Φ(H) (comprising, for example, the number of cuts, distributions of the cuts, distributions of the false cuts, etc.). In a specific embodiment of the present invention only a few of these parameters (number of cuts) are represented by prior models, and the other parameters are implicitly assumed to be equi-probable. Accordingly, in a preferred embodiment, the model of Pr[H] used in accordance with the present invention is relatively simple.

In accordance with the present invention the unconditional distributions for the data Pr[D] in Eqn. (1) does not have to be computed at all because it does not effect the choice of H*. In contrast, in a preferred embodiment of the present invention, a very detailed model is used for the conditional distribution for the data given the chosen parameter values for the hypothesis. One can re-write Eqn. (1) as:

log(Pr[(Φ(H)|D])=Λ+Penalty+Bias, (2)

where Λ=Σⱼ log(Pr[Dⱼ|Φ(H)]) is the likelihood function, Penalty=log Pr(\hat{Φ}(H)) and Bias=−log (Pr[D])=a constant. In these equations Φ(H) corresponds to the parameter set describing the hypothesis and \hat{Φ}(H)⊂(H) a subset of parameters that have a nontrivial prior model. In the following, the symbol H is used for Φ(H), when the context creates no ambiguity.

It should be noted that the bias term in Eqn. (2) has no effect as it is a constant (independent of the hypothesis), and that the penalty term has a discernible effect only when the data set is small. Thus, in a preferred embodiment directed to the use of relatively large data sets, the focus is on the term A which dominates all other terms in the right hand side of Eqn. (2).

Note that the approach based on the Bayesian scheme used in accordance with the present invention enjoys many advantages. For example, one obtains the best possible estimate of the map given the data, subject only to the comprehensiveness of the model Φ(H) used. Further, for a comprehensive model H, estimates of Φ(H) are unbiased and errors converge asymptotically to zero as data size increases. Next, additional sources of error can be modeled simply by adding parameters to Φ(H). It is important for practical applications that estimates of the errors in the result can be computed in a straightforward manner. Advantageously, the algorithm also provides an easy way to compute a quality measure.

As discussed next, however, in general the posterior density, Pr[H|D] used in Eqn. (1) and (2) is multimodal and the prior Pr[Dⱼ|H] does not admit a closed form evaluation (as it is dependent on the orientation and alignment with H). Therefore an iterative sampling technique is developed for the proper evaluation.

In particular, in a preferred embodiment, the method of obtaining accurate restriction maps using the Bayes' formulation above has two parts: (1) a sample hypothesis is taken, and a local search is performed for the most plausible hypothesis in its neighborhood using gradient search techniques; and (2) a global search is used to generate a set of sample hypotheses and filter out all but the ones that are likely to be near plausible hypotheses. The descriptions of the local and global searches performed in accordance with the present invention are described next in that order.

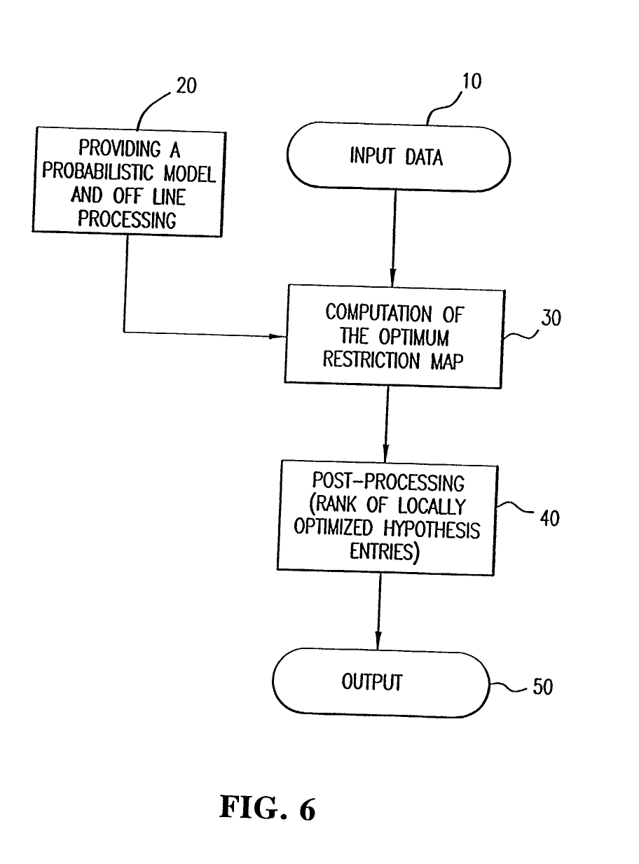

FIG. 6 illustrates in a block-diagram form a preferred embodiment of the method of the present invention. As shown in the figure, at block 10 the method is initiated with input data from the imaging system. This input generally comprises a set of observation vectors (molecules) Dⱼ. With reference to the notations introduced above, at block 20 the method provides a probabilistic model of the data, comprising a hypothesis H of the map of restriction sites, and a model Pr[D|H] of the distribution of the data conditioned on the hypothesis. Also included in this block are various processing routines, used in accordance with the present invention for efficient off-line computation of different output parameters.

At block 30, the method of the present invention combines the input data and the probabilistic model parameters to compute the optimal restriction map hypothesis for the given set of input data. As discussed in detail next, processing 30 comprises in a preferred embodiment two main tasks: (a) conducting a global search over the parameter space for a set of starting hypothesis; and (b) conducting a local search using gradient methods in the vicinity of the selected “seed” hypothesis to obtain the optimal set of parameters for each given hypothesis.

At block 40, in a preferred embodiment the output of processing block 30, expressed in terms of one or more locally optimized hypothesis entries, is sorted under a given “quality of goodness” measure to obtain a final hypothesis, which in a preferred embodiment is the desired accurate restriction map. This map can be stored, displayed, or otherwise processed in block 50. Each of the individual blocks illustrated in FIG. 6 is discussed in detail below.

8. Bayesian Inference—Modeling the Prior Observation Distribution: As noted above, for a relatively large observation space the prior observation distribution Pr[D|H] is the dominant component that determines the accuracy of the restriction maps obtained according to the invention. In a preferred embodiment, Pr[D|H] is modeled considering at least the following categories of errors in the image data: 1) mis-identifying spurious materials in the image as DNA; 2) identifying multiple DNA molecules as one molecule; 3) identifying partial DNA molecules as complete molecules; 4) erring in estimating the sizes of DNA fragments; 5) incompletely digesting the DNA; 6) having cleavage/cut sites visible at locations other than digestion sites; and 7) not knowing the orientation of the DNA molecule being analyzed.

Given these categories, in a preferred embodiment the observation probability distribution Pr[D|H] is modeled as follows:

(1) A molecule on a surface can be read from left to right or right to left. The uncertainty in orientation is modeled as Bernoulli processes, with the probability for each orientation being equal.

(2) The restriction sites on the molecule are determined by a distribution induced by the underlying distribution of the four bases (A, T, C, G) in the DNA. For example, it is assumed that the probability that a particular base (e.g., A) appears at a location “i” is independent of the other bases, although the probabilities are not necessarily identical.

(3) The false cuts appear on the molecule as a Poisson process. This model is based on the simplifying assumption that over a small region Ah on the molecule, the Pr[# False cuts=1 over Δh]=λ_{f} Δh and the Pr[# False cuts≧2 over Δh]=o(Δh).

(4) The fragment size (the size of the molecule between two cuts) is estimated with some loss of accuracy (dependent on the stretching of the molecule, fluorochrome attachments and the image processing algorithm). The measured size is assumed to have a Gaussian distribution.

The modeling process used in accordance with a preferred embodiment is described in more detail next. The following notations will be used to describe the parameters of the independent processes responsible for the statistical structure of the data. Unless otherwise specified, the indices “i,” “j” and “k” are to have the following interpretation:

The index “i” ranges from 1 to N and refers to cuts in the hypothesis; the index “j” ranges from 1 to M and refers to data items (i.e., molecules); the index “k” ranges from 1 to K and refers to a specific alignment of cuts in the hypothesis versus the data.

The main parameters of the Bayesian model used in accordance with a preferred embodiment of the present invention are as follows:

p_{ci}=Probability that the “i”th sequence-specific restriction site in the molecule will be visible as a cut.

σᵢ=Standard deviation of the observed position of the with cut when present and depends on the accuracy with which a fragment can be sized.

λ_{f}=Expected number of false-cuts per molecule observed. Because all sizes will be normalized by the molecule size, this will also designated the false-cuts per unit length.

p_{b}=Probability that the data is invalid (“bad”). In this case, the data item is assumed to have no relation to the hypothesis being tested, and could be an unrelated piece of DNA or a partial molecule with a significant fraction of the DNA missing. The cut-sites (all false) on this data item are assumed to have been generated by a Poisson process with the expected number of cuts=λₙ.

Note that the regular DNA model reduces to the “bad” DNA model for the degenerate situation when p_{ci}→0 and λ_{f}→λₙ. As a result, “bad” DNA molecules cannot be disambiguated from regular DNA molecules if p_{ci}≈0. In practice, p_{ci}>0 and λₙ>λ_{f}, and the degenerate case almost never occurs. The “bad” molecules are recognized by having a disproportionately large number of false cuts.

λₙ=Expected number of cuts per “bad” molecule.

Recall that by Bayes' rule (Eqn. (1)):

Pr[H|D]={Pr[D|H] Pr(H)}/Pr[D]

Assuming that the prior Pr[H] distribution is given in terms of just the number of restriction sites, based on the standard Poisson distribution, the task in accordance with the present invention is to find the “most plausible” hypothesis H by maximizing Pr[D|H].

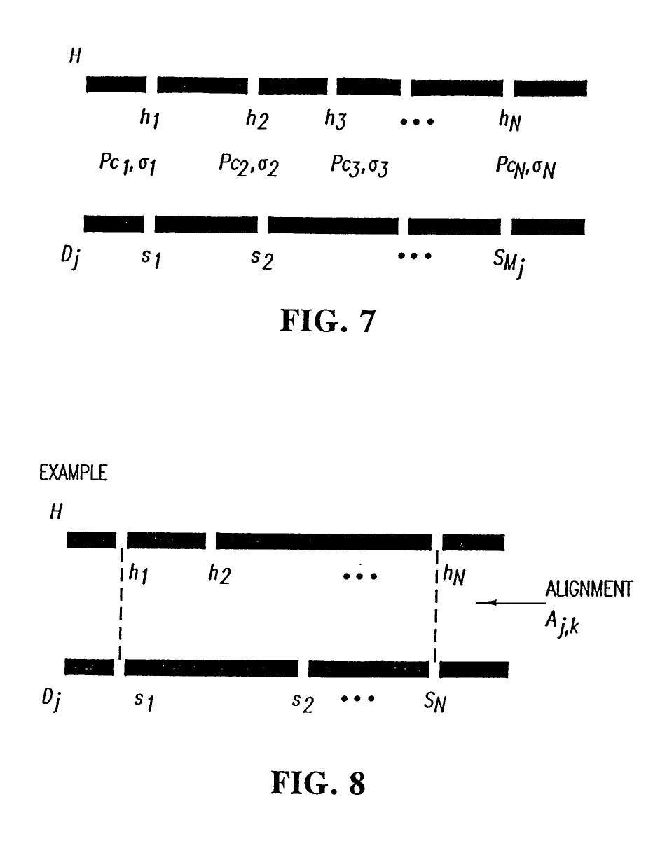

In a preferred embodiment of the present invention, hypothesis H is selected as the final map (a sequence of restriction sites, h₁, h₂, . . . , h_{N}) augmented by certain auxiliary parameters, such as p_{ci}, σᵢ, λ_{f}, etc. Comparing a data item Dⱼ with respect to a hypothesis H, requires consideration of every possible way that Dⱼ could have been generated by H. FIG. 7.illustrates the concept, including certain notations introduced above. In particular, one needs to consider every possible alignment, where the “k”th alignment Aⱼₖ corresponds to a choice of the orientation for Dⱼ, as well as identifying a cut on Dⱼ, with a true restriction site on H, or labeling the cut as a false cut. In the following description Dⱼ^{(A}^{ⱼₖ}⁾ [also abbreviated as Dⱼ^{(k)}], shall denote the interpretation of the “j”th data item with respect to the alignment Aⱼₖ. In a preferred embodiment, each alignment describes an independent process by which Dⱼ could have been generated from H, and therefore the total probability density of Dⱼ is the sum of the probability density of all these alignments, plus the remaining possible derivations (invalid data). As a consequence of the pairwise independence and the preceding discussion, the following holds:

$$Pr [ D \vert H ] = {\prod }_{j}^{M} Pr [ {D}_{j} \vert H ] ,$$

where index j ranges over the data set, and

$${Pr}_{j} ≡ Pr [ {D}_{j} \vert H ] = \frac{1}{2} {\sum }_{k} Pr [ {D}_{j}^{( k )} \vert H , good ] Pr [ good ] + \frac{1}{2} {\sum }_{k} Pr [ {D}_{j}^{( k )} \vert H , bad ] Pr [ bad ]$$

where index “k” ranges over the set of alignments.

In the above equation, Pr[Dⱼ^{(k)}|H,good] (denoted for simplicity as Prⱼₖ) is the probability density of model Dⱼ being derived from model H and corresponding to a particular alignment of cuts (denoted, Aⱼₖ). The set of alignments include alignments for both orientations, hence each alignment has a prior probability of ½. If Dⱼ is bad, the model corresponds to H with p_{ci}→0 and λ_{f}→λₙ. The qualifier “good” for the hypothesis H is omitted, when it is clear from the context.

Thus, in the example shown in FIG. 8, for a given hypothesis H, the conditional probability density that the “j”th data item Dⱼ with respect to alignment Aⱼₖ (i.e., Dⱼ^{(k)}) could have occurred is given by the following expression:

$${Pr}_{ij} = {p}_{c1} \frac{{e}^{- {( {s}_{1} - {h}_{1} )}^{2}} / 2 {σ}_{2}^{2}}{\sqrt{2 π} {σ}_{1}} \times ( 1 - {P}_{c2} ) \times {λ}_{f} {e}^{- {λ}_{f}} \times … \times {p}_{cN} \frac{{e}^{- {( {s}_{N} - {h}_{N} )}^{2} / 2 {σ}_{N}^{2}}}{\sqrt{2 π} {σ}_{N}}$$

The following notations are used next in the most general case considered:

N≡Number of cuts in the hypothesis H.

hᵢ≡The “i”th cut location on H.

Mⱼ≡Number of cuts in the data Dⱼ.

Kⱼ=Number of possible alignments of the data/evidence Dⱼ against the hypothesis H (or its reversal, the flipped alignment H^{R}).

sᵢⱼₖ=The cut location in Dⱼ matching the cut hi in H, given the alignment Aⱼₖ. In case such a match occurs, this event is denoted by an indicator variable mᵢⱼₖ taking the value 1.

mⱼₖ=An indicator variable, taking the value 1 if the cut sᵢⱼₖ in Dⱼ matches a cut hᵢ in the hypothesis H, given the alignment Aⱼₖ. It takes the value 0, otherwise.

Fⱼₖ=Number of false (non-matching) cuts in the data Dⱼ for alignment Aⱼₖ, that do not match any cut in the hypothesis H. Thus

$${F}_{jk} = {M}_{j} - {\sum }_{i = 1}^{N} {m}_{ijk}$$

The number of missing cuts is thus:

$${\sum }_{i = 1}^{N} ( 1 - {m}_{ijk} ) = N - {\sum }_{i = 1}^{N} {m}_{ijk}$$

By an abuse of notation, the indices “j” and “k” may be omitted, if from the context it can be uniquely determined which data Dⱼ and alignment Aⱼₖ are being referenced. Note that a fixed alignment Aⱼₖ can be described uniquely by marking the cuts on Dⱼ by the labels T (for true cut) and F (for false cut) and by further augmenting each true cut by the identity of the cut hᵢ of the hypothesis H. From this information, mᵢⱼₖ, sᵢⱼₖ, Fⱼₖ, etc. can all be determined uniquely. Let the cuts of Dⱼ be (s₁, s₂, . . . , s_{Mj}). Also, let the event Eᵢ denote the situation in which there is a cut in the infinitesimal interval (sᵢ−Δx/2, sᵢ+Δx/2). This yields:

$$\begin{matrix}Pr [ {D}_{j}^{( k )} \vert H , good ] Δ {x}_{1} … Δ {x}_{Mj} = Pr [ {D}_{j}^{( k )} \vert H , good ] {( Δ x )}^{Mj} \\ = Pr [ {E}_{1} , … , {E}_{Mj} , {A}_{jk} \vert H , good ] \\ = Pr [ {E}_{1} , … , {E}_{Mj} , {A}_{jk} \vert {m}_{ijk} , {M}_{j} , H , good ] \times \\ Pr [ {m}_{ijk} , {M}_{j} \vert H , good ] \\ = Pr [ {E}_{1} , {A}_{jk} \vert {m}_{ijk} , {M}_{j} , H , good ] \times \\ Pr [ {E}_{2} , {A}_{jk} \vert {E}_{1} , {m}_{ijk} , {M}_{j} , H , good ] \times … \times \\ Pr [ {E}_{α} , {A}_{jk} \vert {E}_{1} , … , {E}_{α - 1} , {m}_{ijk} , {M}_{j} , H , good ] \times … \times \\ Pr [ {E}_{Mj} , {A}_{jk} \vert {E}_{1} , … , {E}_{Mj - 1} , {m}_{ijk} , {M}_{j} , H , good ] \times \\ Pr [ {m}_{ijk} , {M}_{j} \vert H , good ]\end{matrix}$$

Note also the following:

$$\begin{matrix}Pr [ {m}_{ijk} , {M}_{j} \vert H , good ] = [ {\prod }_{i = 1}^{N} ( {p}_{ci} {m}_{ijk} + ( 1 - {p}_{ci} ) ( 1 - {m}_{ijk} ) ) ] \times \\ {e}^{- {λ}_{f}} {λ}_{f}^{{F}_{jk}} / {F}_{jk} ! \\ = [ {\prod }_{i = 1}^{N} {{p}_{ci}^{{m}_{ijk}} ( 1 - {p}_{ci} )}^{( 1 - {m}_{ijk} )} ] \times \\ {e}^{- {λ}_{f}} {λ}_{f}^{{F}_{jk}} / {F}_{jk} !\end{matrix}$$

For the event E_{α} there are two possible situations to be considered:

(1) s_{α} is a false cut and the number of false cuts among s₁, . . . , s_{α-1} is β: Pr[E_{α}Aⱼₖ|E₁, . . . , E_{α-1}, mᵢⱼₖ, Mⱼ, H, good]=(Fⱼₖ−β)Δx.

(2) s_{α}=sᵢⱼₖ is a true cut and hᵢ is the cut in H associated with it:

$$\begin{matrix}Pr [ {E}_{1} , … , {E}_{{M}_{j}} , {A}_{jk} \vert {m}_{ijk} , {M}_{j} , H , good ] = {\prod }_{i = 1}^{N} {( \frac{{e}^{- {( {s}_{ijk} - {h}_{i} )}^{2} / 2 {σ}_{i}^{2}}}{\sqrt{2 π} {σ}_{i}} Δ x )}^{{m}_{ijk}} \times {F}_{jk} ! {( Δ x )}^{{F}_{jk}} \\ = {{F}_{jk} ! \prod }_{i = 1}^{N} {( \frac{{e}^{- {( {s}_{ijk} - {h}_{i} )}^{2} / 2 {σ}_{i}^{2}}}{\sqrt{2 π} {σ}_{i}} )}^{{m}_{ijk}} {( Δ x )}^{{M}_{j}}\end{matrix}$$

$${}_{j}^{( k )} \vert H , good \vert = [ {\prod }_{i = 1}^{N} {( {p}_{{c}_{i}} \frac{{e}^{- {( {s}_{ijk} - {h}_{i} )}^{2} / 2 {σ}_{i}^{2}}}{\sqrt{2 π} {σ}_{i}} )}^{{m}_{ijk}} {( 1 - {p}_{{c}_{i}} )}^{( 1 - {m}_{ijk} )} ] \times {e}^{- λ}$$

$$\text{Thus} , \\ Pr [ {E}_{α} , {A}_{jk} \vert {E}_{1} , … , {E}_{α - 1} , {m}_{ijk} , {M}_{j} , H , good ] = \frac{{e}^{- {( {S}_{ijk} - {h}_{i} )}^{2} / 2 {σ}_{i}^{2}}}{\sqrt{2 π} {σ}_{i}} Δ x$$

By an identical argument it can be seen that the only alignments relevant for the “bad” molecules correspond to the situation when all cuts in Dⱼ are labeled false, and for each of two such alignments,

Pr[Dⱼ^{(k)}|H,bad]=e^{-λ}^{ₙ}λₙ^{M}^{ⱼ}

Accordingly, in a preferred embodiment of the present invention the log-likelihood can then be computed as follows:

Λ≡Σⱼ log Pr[Dⱼ|H].

In particular,

$$= {\sum }_{j} log [ {p}_{b} {e}^{- {λ}_{n}} {λ}_{n}^{{M}_{j}} + \frac{( 1 - {p}_{b} )}{2} {\sum }_{k} {Pr}_{jk} ] = {\sum }_{j} log [ {p}_{b} {e}_{j} + ( 1 - {p}_{b} ) \qquad ( 3 )$$

where by definition, p_{b} is the probability that the data is invalid (“bad”), and

$${e}_{j} ≡ {e}^{- {λ}_{n}} {λ}_{n}^{{M}_{j}} ; {d}_{j} ≡ \frac{( {\sum }_{k} {Pr}_{jk} )}{2}$$

In a preferred embodiment of the present invention, Eqn. (3) for the log-likelihood function is used along with the model of the hypothesis space distribution (considered next) to model the posterior distributions Pr[H|D] for a given observation space D. As known in the art, for a given hypothesis H, taking derivatives with respect to the model parameters and solving the resulting equations gives the hypothesis H* that corresponds to the desired output restriction map.

In a specific embodiment of the present invention, the prior distribution in the hypotheses space Pr[H] (and consequently the penalty term in Eqn. (2) above) has a simple model that only depends on the number of restriction sites N. The model implicitly assumes that all hypotheses with same number of cuts are equi-probable, independent of the cut location. Thus, given a k-cutter enzyme (e.g., normally six-cutters like EcoR I in a specific embodiment), the probability that the enzyme cuts at any specific site in a sufficiently long clone is given by:

pₑ=(¼)^{k}.

Thus, if a clone is of length G base pairs and the expected number of restriction sites in the clone λₑ=G pₑ, then the probability that the clone has exactly N restriction cuts is given by:

Pr[# restriction sites=N|enzyme, e and clone of length G]≡exp{−λₑ}λₑⁿ/N!.

This expression is based on the assumption that all four bases ∈{A, T, C, G} occur in the clone with equal probability=¼. However, it is known that the human genome is CG-poor (i.e., Pr[C]+Pr[G]=0.32<Pr[A]+Pr[T]=0.68). Therefore, in a preferred embodiment of the present invention a more realistic model is used to provide a better estimation for pₑ, given by the expression:

pₑ=(0.16)^{#CG}(0.34)^{#AT},

where “#CG” denotes the number of C or G bases in the restriction enzyme cleavage site similarly “#AT” denotes the number of A or T bases in the restriction enzyme cleavage site.

9. Local Search Algorithm: Assume first that a hypothesis is defined over the parameter space and the task is to define the best, i.e., most plausible, restriction map given the input observation space. In order to find the most plausible restriction map, the cost function derived above is optimized with respect to the following parameters:

Cut Sites=h₁, h₂, . . . , h_{N}

Cut Rates=p_{c1}, p_{c2}, . . . , p_{cN}

Std. Dev. of Cut Sites=σ₁, σ₂, . . . , σ_{N}

Auxiliary Parameters=p_{b}, λ and λₙ.

Let any of these parameters be denoted by θ. As known in the art, with reference to Eqn. (2) above, the optimal solution with respect to each individual parameter θ is found using the equation (4),

$$\frac{\partial Λ}{\partial θ} = 0 , \qquad ( 4 )$$

which gives the extreme point of Λ with respect to the individual parameter θ.

Next, the computation of each of the individual parameters in accordance with the present invention is considered separately.

Case 1: θ→p_{b}:

Taking the first partial derivative of the likelihood function with respect to p_{b} gives:

$$\frac{\partial Λ}{\partial {p}_{b}} = {\sum }_{j} \frac{( {e}_{j} - {d}_{j} )}{{p}_{b} {e}_{j} + ( 1 - {p}_{b} ) {d}_{j}} \qquad ( 5 )$$

where p_{b} is the probability that the data is invalid, and eⱼ and dⱼ are as defined in Eqn. (3). Taking the second partial derivative gives:

$$\frac{{\partial }^{2} Λ}{\partial {p}_{b}^{2}} = - {\sum }_{j} \frac{{( {e}_{j} - {d}_{j} )}^{2}}{{[ {p}_{b} {e}_{j} + ( 1 - {p}_{b} ) {d}_{j} ]}^{2}} \qquad ( 6 )$$

According to the preferred embodiment of the invention, Λ can be optimized iteratively to estimate the best value of p_{b} by means of the following application of Newton's equation:

$${p}_{b} := {p}_{b} - \frac{\partial Λ / \partial {p}_{b}}{{\partial }^{2} Λ / \partial {p}_{b}^{2}}$$

where the first and second partial derivatives are as indicated above. The above expression is used for iterative optimization. Iterative techniques for function optimization are known in the art and need not be considered in detail.

Case 2: θ→λₙ:

The expected number of cuts per “bad” molecule is simply estimated to be the average number of cuts. Note that,

$$\frac{\partial Λ}{\partial {λ}_{n}} = {\sum }_{j} \frac{{p}_{b} {e}_{j} ( {M}_{j} / {λ}_{n} - 1 )}{{p}_{b} {e}_{j} + ( 1 - {p}_{b} ) {d}_{j}}$$

should be zero at the local maxima. Thus a good approximation is obtained by taking:

$${\sum }_{j} ( \frac{{M}_{j}}{{λ}_{n}} - 1 ) \approx 0$$

leading to the update rule:

$${λ}_{n} := \frac{{\sum }_{j} {M}_{j}}{{\sum }_{j} 1} = \frac{{\sum }_{j} {M}_{j}}{\text{Total number of molecules}}$$

Thus, in the preferred embodiment, λₙ is simply the average number of cuts per molecule.

Case 3: θ→hᵢ, p_{ci}, σᵢ(i=1, . . . , N), or λ:

Unlike in the previous two cases, these parameters are in the innermost section of the probability density expression and computing any of these gradients will turn out to be computationally comparable to evaluating the entire probability density. In this case:

$$\frac{\partial Λ}{\partial θ} = {\sum }_{j} \frac{1}{{Pr}_{j}} ( \frac{1 - {p}_{b}}{2} {\sum }_{k} {Pr}_{jk} {χ}_{jk} ( θ ) ) ,$$

where Prⱼ≡Pr[Dⱼ|H]

and where:

$${χ}_{jk} ( θ ) ≡ [ \frac{{F}_{jk}}{{λ}_{f}} \frac{\partial {λ}_{f}}{\partial θ} - \frac{\partial {λ}_{f}}{\partial θ} ] + {\sum }_{i = 1}^{N} [ \frac{{m}_{ijk}}{{p}_{ci}} \frac{\partial {p}_{ci}}{\partial θ} - \frac{1 - {m}_{ijk}}{1 - {p}_{ci}} \frac{\partial {p}_{ci}}{\partial θ} ] + {\sum }_{i = 1}^{N} {m}_{ijk} [ \frac{\partial }{\partial θ} ( \frac{- {( {s}_{ijk} - {h}_{i} )}^{2}}{2 {σ}_{i}^{2}} ) - \frac{1}{{σ}_{i}} \frac{\partial {σ}_{i}}{\partial θ} ]$$

For convenience, πⱼₖ is defined as:

$${π}_{jk} ≡ ( \frac{1 - {p}_{b}}{2} ) \frac{{Pr}_{jk}}{{Pr}_{j}}$$

as the relative probability density of the alignment Aⱼₖ for data item Dⱼ.

Thus, the expression for the partial derivative with respect to θ simplifies to

$$\frac{\partial Λ}{\partial θ} = {\sum }_{j} {\sum }_{k} {π}_{jk} {χ}_{jk} ( θ )$$

Before examining the updating formula for each parameter optimization, the following notations are introduced for future use. In a preferred embodiment, the quantities defined below are efficiently accumulated for a fixed value of the set of parameters.

Ψ₀ᵢ≡ΣⱼΣₖπⱼₖmᵢⱼₖ≡Expected number of cuts matching hᵢ

Ψ₁ᵢ≡ΣⱼΣₖπⱼₖmᵢⱼₖsᵢⱼₖ≡Sum of cut locations matching hᵢ

Ψ₂ᵢ≡ΣⱼΣₖπⱼₖmᵢⱼₖsᵢⱼₖ²≡Sum of square of cut locations matching hᵢ.

μ_{g}≡ΣⱼΣₖπⱼₖ≡Expected number of “good” molecules.

γ_{g}≡ΣⱼΣₖπⱼₖMⱼ≡Expected number of cuts in “good” molecules.

We note here that the Ψ's can all be computed efficiently using a simple updating rule that modifies the values with one data item Dⱼ (i.e., one molecule) at a time. This rule can then be implemented using a dynamic programming recurrence equation (described below).

Case 3A: θ→hᵢ:

Note that

$$\begin{matrix}θ ≡ {h}_{i} ⇒ {χ}_{jk} ( {h}_{i} ) = {m}_{ijk} ( {s}_{ijk} - {h}_{i} ) / {σ}_{i}^{2} \\ ⇒ \frac{\partial Λ}{\partial {h}_{i}} = {\sum }_{j} {\sum }_{k} {π}_{jk} {m}_{ijk} ( {s}_{ijk} - {h}_{i} ) / {σ}_{i}^{2}\end{matrix}$$

$$\text{Thus} , \\ \frac{\partial Λ}{\partial {h}_{i}} = \frac{1}{{σ}_{i}^{2}} ( {Ψ}_{1 i} - {h}_{i} {Ψ}_{0 i} )$$

Although Ψ's depend on the location hᵢ, they vary rather slowly as a function of hᵢ. Hence, a feasible update rule for hᵢ in accordance with the present invention is:

hᵢ=Ψ₁ᵢ/Ψ₀ᵢ (7)

Thus the updated value of hᵢ is simply the “average expected value” of all the sᵢⱼₖ's that match the current value of hᵢ.

Case 3B: θ→p_{ci}:

Note that

$$\begin{matrix}θ ≡ {p}_{ci} ⇒ {χ}_{jk} ( {p}_{ci} ) = \frac{{m}_{ijk}}{{p}_{ci}} - \frac{1 - {m}_{ijk}}{1 - {p}_{ci}} \\ ⇒ \frac{\partial Λ}{\partial {p}_{ci}} = {\sum }_{j} {\sum }_{k} {π}_{jk} ( \frac{{m}_{ijk}}{{p}_{ci}} - \frac{1 - {m}_{ijk}}{1 - {p}_{ci}} )\end{matrix}$$

$$\text{Thus:}$$

$$\frac{\partial Λ}{\partial {p}_{ci}} = \frac{{Ψ}_{0 i}}{{p}_{ci}} - \frac{{μ}_{g} - {Ψ}_{0 i}}{1 - {p}_{ci}}$$

Arguing as before, the following feasible update rule for p_{ci} can be used:

p_{ci}:=Ψ₀ᵢ/μ_{g}. (8)

Thus, in a preferred embodiment of the present invention, p_{ci} is the fraction of the “good” molecules that have a matching cut at the current value of hᵢ.

Case 3C: θ→σᵢ:

Note that,

$$\begin{matrix}θ ≡ {σ}_{i} ⇒ {χ}_{jk} ( {σ}_{i} ) = {m}_{ijk} ( \frac{{( {s}_{ijk} - {h}_{i} )}^{2}}{{σ}_{i}^{3}} - \frac{1}{{σ}_{i}} ) \\ ⇒ \frac{\partial Λ}{\partial {σ}_{i}} = {\sum }_{j} {\sum }_{k} {π}_{jk} {m}_{ijk} ( \frac{{( {s}_{ijk} - {h}_{i} )}^{2}}{{σ}_{i}^{3}} - \frac{1}{{σ}_{i}} )\end{matrix}$$

$$\text{Thus:}$$

$$\frac{\partial Λ}{\partial {σ}_{i}} = \frac{1}{{σ}_{i}^{3}} ( {Ψ}_{2 i} - 2 {h}_{i} {Ψ}_{1 i} + {h}_{i}^{2} {Ψ}_{0 i} - {σ}_{i}^{2} {Ψ}_{01} ) . \\ {σ}_{i}^{2} := \frac{( {Ψ}_{2 i} - 2 {h}_{i} {Ψ}_{1 i} + {h}_{i}^{2} {Ψ}_{0 i} )}{{Ψ}_{0 i}}$$

This gives the following feasible update rule for σᵢ²:

Using the estimate for hᵢ (Eqn. 4), the immediately preceding equation simplifies to:

$${σ}_{i}^{2} := \frac{{Ψ}_{2 i}}{{Ψ}_{0 i}} - {( \frac{{Ψ}_{1 i}}{{Ψ}_{0 i}} )}^{2} \qquad ( 9 )$$

Accordingly, in a preferred embodiment of the present invention, the model parameters are simply the variance of all the sᵢⱼₖ's that match the current value of hᵢ.

Case 3D: θ→λ:

Note that

$$\begin{matrix}θ ≡ {λ}_{f} ⇒ {χ}_{jk} ( {λ}_{f} ) = \frac{{F}_{jk}}{{λ}_{f}} - 1 = \frac{{M}_{j} - {\sum }_{i} {m}_{ijk}}{{λ}_{f}} - 1 \\ ⇒ \frac{\partial Λ}{\partial {λ}_{f}} = {\sum }_{j} {\sum }_{k} {π}_{jk} ( \frac{{M}_{j} - {\sum }_{i} {m}_{ijk}}{{λ}_{f}} - 1 ) \\ = \frac{{γ}_{g} - {\sum }_{i} {Ψ}_{0 i}}{{λ}_{f}} - {μ}_{g}\end{matrix}$$

This gives the following feasible update rule for λ_{f}:

$${λ}_{f} := \frac{{γ}_{g}}{{μ}_{g}} - {\sum }_{i} \frac{{Ψ}_{0 i}}{{μ}_{g}} \qquad ( 10 )$$

Accordingly, in a preferred embodiment of the present invention the model parameter is computed as the average number of unmatched cuts per “good” molecule. (Note that the molecules are already normalized to unit length.)

Case 3E: θ→p_{c}=p_{c1}= . . . =p_{cN} (Constrained):

Note that:

$$\frac{\partial Λ}{\partial {p}_{c}} = {\sum }_{j} {\sum }_{k} {\sum }_{i = 1}^{N} {π}_{jk} ( \frac{{m}_{ijk}}{{p}_{c}} - \frac{1 - {m}_{ijk}}{1 - {p}_{c}} )$$

Thus, the update rule for this case is:

$${p}_{c} := \frac{{\sum }_{i} {Ψ}_{0 i} / N}{{μ}_{g}} \qquad ( 11 )$$

Case 3F: θ→σ=σ₁= . . . =σ_{N} (Constrained):

In this case:

$$\frac{\partial Λ}{\partial σ} = {\sum }_{j} {\sum }_{k} {\sum }_{i = 1}^{N} {π}_{jk} {m}_{ijk} ( \frac{{( {s}_{ijk} - {h}_{i} )}^{2}}{{σ}^{3}} - \frac{1}{σ} )$$

The update equation for this case therefore is:

$${σ}^{2} := \frac{{\sum }_{i} ( {Ψ}_{2 i} - {Ψ}_{1 i}^{2} / {Ψ}_{0 i} )}{{\sum }_{i} {Ψ}_{0 i}} \qquad ( 12 )$$

Equations (3)-(12) above define the local search algorithm used in a specific embodiment of the present invention to determine the most plausible hypothesis in the neighborhood of a sample hypothesis H, using gradient search techniques. In the next section, an update algorithm using dynamic programming is disclosed. This algorithm can be used to determine the desired quantities in a computationally efficient way.

10. Update Algorithm—Dynamic Programming: As seen in the preceding section, in each update step of the gradient search, one needs to compute the new values of the parameters based on the old values of the parameters, which affect the “moment functions”: Ψ₀ᵢ, Ψ₁ᵢ, Ψ₂ᵢ, μ_{g} and γ_{g}. For ease in expressing the computation, the following additional auxiliary expressions are used below:

$$\begin{matrix}{P}_{j} ≡ {\sum }_{k} ( \frac{{Pr}_{jk}}{{e}^{- {λ}_{f}}} ) \\ {W}_{ij} ≡ {\sum }_{k} ( \frac{{Pr}_{jk} {m}_{ijk}}{{e}^{- {λ}_{f}}} ) \\ {SUM}_{ij} ≡ {\sum }_{k} ( \frac{{Pr}_{jk} {m}_{ijk} {s}_{ijk}}{{e}^{- {λ}_{f}}} ) \\ {SQ}_{ij} ≡ {\sum }_{k} ( \frac{{Pr}_{jk} {m}_{ijk} {s}_{ijk}^{2}}{{e}^{- {λ}_{f}}} )\end{matrix} \qquad ( 13 )$$

One motivation for this formulation is to avoid having to compute e^{-λ} repeatedly, because this is a relatively expensive computation. The original moment function can now be computed as follows:

$$\begin{matrix}{Pr}_{j} = ( \frac{1 - {p}_{b}}{2} ) {e}^{- {λ}_{f}} \times {P}_{j} + {p}_{b} {e}_{j} \\ {Ψ}_{0 i} = ( \frac{1 - {p}_{b}}{2} ) {e}^{- {λ}_{f}} {\sum }_{j} \frac{{W}_{ij}}{{Pr}_{j}} \\ {Ψ}_{1 i} = ( \frac{1 - {p}_{b}}{2} ) {e}^{- {λ}_{f}} {\sum }_{j} \frac{{SUM}_{ij}}{{Pr}_{i}} \\ {Ψ}_{2 i} = ( \frac{1 - {p}_{b}}{2} ) {e}^{- {λ}_{f}} {\sum }_{j} \frac{{SQ}_{ij}}{{Pr}_{j}} \\ {μ}_{g} = ( \frac{1 - {p}_{b}}{2} ) {e}^{- {λ}_{f}} {\sum }_{j} \frac{{P}_{j}}{{Pr}_{j}} \\ {γ}_{g} = ( \frac{1 - {p}_{b}}{2} ) {e}^{- {λ}_{f}} {\sum }_{j} \frac{{M}_{f} {P}_{j}}{{Pr}_{j}}\end{matrix} \qquad ( 14 )$$

The definitions for Pⱼ, Wᵢⱼ, SUMᵢⱼ and SQᵢⱼ involve all alignments between each data element Dⱼ and the hypothesis H. This number is easily seen to be exponential in the number of cuts N in the hypothesis H, even if one excludes such physically impossible alignments as the ones involving cross-overs (i.e., alignments in which the order of cuts in H and Dⱼ are different). First, consider Pⱼ:

$$\begin{matrix}{P}_{j} ≡ {\sum }_{k} ( \frac{{Pr}_{jk}}{{e}^{- {λ}_{f}}} ) \\ = {\sum }_{k} [ {\prod }_{i = 1}^{N} {( {p}_{ci} \frac{{e}^{- {( {h}_{i} - {s}_{ijk} )}^{2} / 2 {σ}_{i}^{2}}}{\sqrt{2 π} {σ}_{i}} )}^{{m}_{ijk}} \times {\prod }_{i = 1}^{n} {( 1 - {p}_{ci} )}^{1 - {m}_{ijk}} \times {λ}_{f}^{{F}_{jk}} ]\end{matrix}$$

What follows is a description of a set of recurrence equations used in the preferred embodiment for computing the values for all alignments efficiently. The set of alignments computed are for the cuts 1, . . . , Mⱼ of Dⱼ, mapped against the hypothesized cuts 1, . . . , N. The recurrence equations are defined in terms of:

P_{q,r}≡Pⱼ(s_{q}, . . . , s_{Mj}; hᵣ, . . . , h_{N}),

which is the probability density of all alignments for the simpler problem in which cuts s₁, . . . , s_{q-1} are missing in the data Dⱼ, and the cuts h₁, . . . , hᵣ₋₁ are missing in the hypothesis H. In this instance:

$$\begin{matrix}{P}_{j} ≡ {P}_{1 , 1} \\ {P}_{q , r} ≡ {λ}_{f} {P}_{q + 1 , r} + {\sum }_{t = r}^{N} {P}_{q + 1 , t + 1} [ {\prod }_{t = r}^{t - 1} ( 1 - {p}_{ci} ) ] {p}_{ci} \frac{{e}^{- {( {h}_{t} - {s}_{q} )}^{2} / 2 {σ}_{i}^{2}}}{\sqrt{2 π} {σ}_{t}}\end{matrix} \qquad ( 15 )$$

where 1≦q≦Mⱼ and 1≦r≦N+1.

Eqn. (15) follows from a nested enumeration of all possible alignments. The recurrence terminates in P_{Mj+1,r}, which represents Pⱼ if all cuts in Dⱼ were missing and cuts h₁, . . . , hᵣ₋₁ in H were missing:

$${P}_{{M}_{j} , r} = {\sum }_{i = r}^{N} ( 1 - {p}_{ci} ) \qquad ( 16 )$$

Thus the total number of terms P_{q,r} to be computed is bounded from above by (Mⱼ+1) (N+1) where Mⱼ is the number of cuts in data molecule Dⱼ and N is the number cuts in H. Each term can be computed in descending order of “q” and “r” using Equations (15) and (16). The time complexity associated with the computation of P_{q,r} is O(N−r) in terms of the arithmetic operations.

Note also that the Eqn. (15) can be written in the following alternative form:

$$\begin{matrix}{P}_{j} ≡ {P}_{1 , 1} \\ {P}_{q , r} ≡ {λ}_{f} {P}_{q + 1 , r} + {P}_{q + 1 , r + 1} {p}_{ci} \frac{{e}^{- {( {h}_{t} - {s}_{q} )}^{2} / 2 {σ}_{t}^{2}}}{\sqrt{2 π} {σ}_{t}} + \\ ( 1 - {p}_{cr} ) [ {p}_{q , r + 1} - {λ}_{f} {P}_{q + 1 , r + 1} ]\end{matrix} \qquad ( 17 )$$

where 1≦q≦Mⱼ and 1≦r≦N+1.

Thus, by computing P_{q,r} in descending order of “r,” only two new terms and one new product, (1−p_{cr}) in Eqn. (17), needs be to be computed for each P_{q,r}. With this modification, the overall time complexity of the iterative computation used in accordance with the present invention reduces to O(Mⱼ N).

The complexity can be improved further by taking advantage of the fact that the exponential term is negligibly small unless hₜ and s_{q} are sufficiently close (e.g., |hₜ−s_{q}|≦3 σₜ. For any given value of q, only a small number of hₜ will be close to s_{q}. For a desired finite precision only a small constant fraction of hₜ's will be sufficiently close to s_{q} to require that the term with the exponent be included in the summation. It should be noted that in practice, even a precision of 10⁻¹⁰ will only require 3—5 terms to be included with σ around 1%.

Note, however, that even with this optimization in the computation for Eqn. (15), the computation of P_{q,r} achieves no asymptotic improvement in the time complexity, because P_{q,r} with consecutive “r” can be computed with only two new terms, as noted earlier. However, for any given “q,” only for a few “r” values are both of these additional terms non-negligible. The range of “r” values (say, between rₘᵢₙ and rₘₐₓ) for which the new terms with (exp{−(hᵣ−s_{q})²/2σₜ²}) is significant and can be precomputed in a table indexed by q=1, . . . , Mⱼ. For r>rₘₐₓ all terms in the summation are negligible. For r<rₘᵢₙ the new exponential term referred to previously is negligible. In both cases, the expression for P_{q,r} can be simplified:

$${P}_{q , r} = \vert \begin{matrix}{λ}_{f} {P}_{q + 1 , r} & \text{if} r > {r}_{max} [ q ] ; \\ {λ}_{f} {P}_{q + 1 , r} + ( 1 - {P}_{cr} ) ( {P}_{q , r + 1} - {λ}_{f} {P}_{q + 1 , r + 1} ) , & \text{if} r < {r}_{min} [ q ]\end{matrix} \qquad ( 18 )$$

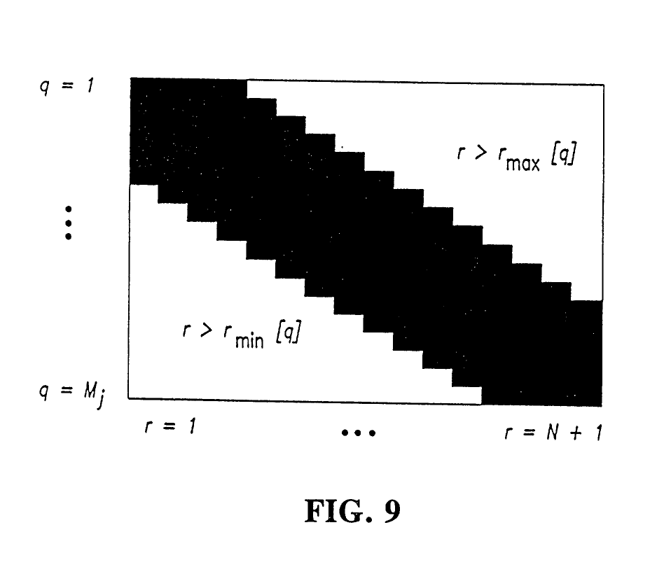

Because both rₘᵢₙ[q] and rₘₐₓ[q] are monotonically non-decreasing functions of “q,” the (q,r) space divides as shown in FIG. 9. Of course, the block diagonal pattern need not be as regular as shown and will differ for each data molecule Dⱼ.

Note again that the ultimate object is to compute P_{1,1}. Terms P_{q,r+1} with r>rₘₐₓ[q], cannot influence any term P_{q′,r′} with r′≦r (see Eqn. (15)). Therefore, any term P_{q,r+1} with r>rₘₐₓ[q] cannot influence P_{1,1} as is readily seen by a straightforward inductive argument. Therefore, all such terms need not be computed at all.

For r<rₘₐₓ[q], these terms are required but need not be computed since they always satisfy the following identity:

P_{q,r}=(1−P_{Cr}}) P_{q,r+1}, r<rₘᵢₙ[q].

This follows from Eqns. (16) and (18) by induction on “q.” These terms can then be generated on demand when the normal recurrence (Eqn. (15)) is computed and whenever a term P_{q+1,r} is required for which r<rₘᵢₙ[q+1], provided terms are processed in descending order of “r.”

Thus, the effective complexity of the algorithm used in the preferred embodiment of the present invention is O(Mⱼrₘₐₓ−rₘᵢₙ+2)). Becuase rₘₐₓ−rₘᵢₙ+2 is proportional for a given precision to ┌(σN+1)┐ (where σ is an upper bound on all the σₜ values), it can be seen that the time complexity for a single molecule Dⱼ is O(σ Mⱼ N). Summing over all molecules Dⱼ, the total time complexity of the algorithm in accordance with the present invention is O(σ M N), where M=ΣⱼMⱼ. The space complexity is trivially bounded by O(Mₘₐₓ N) where Mₘₐₓ=maxⱼMⱼ.

Essentially the same recurrence equations can be used to compute the quantities Wᵢⱼ, SUMᵢⱼ and SQᵢⱼ, since these 3N quantities sum up the same probability densities Prⱼₖ weighted by mᵢⱼₖ, mᵢⱼₖsᵢⱼₖ or mᵢⱼₖsᵢⱼₖ² respectively. The difference is that the termination of the recurrence (Eqn.(15)) is simply P_{Mj+1,r}=0, whereas the basic recurrence equation (Eqn. (15)) contains an additional term corresponding to the mᵢⱼₖ times the corresponding term in the recurrence equation. For example:

$${SUM}_{ij} ≡ {SUM}_{i , 1 , 1} \text{and} \\ {SUM}_{i , q , r} ≡ {λ}_{f} {SUM}_{i , q + 1 , r} + {\sum }_{t = r}^{N} {SUM}_{i , q + 1 , t + 1} [ {\prod }_{j = r}^{t - 1} ( 1 - {p}_{cj} ) ] {p}_{ct} \frac{{ⅇ}^{- {( {h}_{t} - {s}_{q} )}^{2} / 2 {σ}_{t}^{2}}}{\sqrt{2 π} {σ}_{t}} + {I}_{i \geq r} {s}_{q} {P}_{q + 1 , i + 1} [ {\prod }_{j = r}^{i - 1} ( 1 - {p}_{cj} ) ] {p}_{ci} \frac{{ⅇ}^{- {( {h}_{i} - {s}_{q} )}^{2} / 2 {σ}_{i}^{2}}}{\sqrt{2 π} {σ}_{i}} \qquad ( 19 )$$

where 1≦q≦Mⱼ and 1≦r≦N+1, and the expression:

I_{i≧r}≡(i≧r? 1:0) is a shorthand for 1, if i≧r; and 0 otherwise.

Note that the new term is only present if i≧r, and as before need only be computed if the corresponding exponent is significant, i.e., “i” lies between rₘᵢₙ[q] and rₘₐₓ[q]. This term is the only nonzero input term in the recurrence because the terminal terms are zero. This recurrence is most easily derived by noting (from Eqns. (3) and (13)) that the sum of products form of SUMᵢⱼ, can be derived from that of Pⱼ by multiplying each product term with hᵢ−s_{q} in any exponent by s_{q}, and deleting any term without hᵢ in the exponent. Because each product term contains at most one exponent with hi, this transformation can also be applied to the recurrence form for Pⱼ (Eqn. (15)), which is just a different factorization of the original sum of products form. The result is Eqn. (19).

The corresponding derivation for Wᵢⱼ and SQᵢⱼ is the same, except that the s_{q} is replaced by 1 or s_{q}² respectively. If the recurrences for these 3N quantities are computed in parallel with the probability density Pⱼ, the cost of the extra term is negligible, so the overall cost of computing both the probability density Pⱼ and its gradients is O(σ M N²). The cost of conversion Eqns. (14) is also negligible in comparison. Moreover this can be implemented as a vectorized version of the basic recurrence with vector size 3N+1 to take advantage of either vector processors or superscalar pipelined processors. Also, as an aside, if 3N is significantly greater than the average width a M of the dynamic programming block diagonal matrix shown in FIG. 9 then a standard strength reduction can be applied to the vectorized recurrence equations trading the 3N vector size for a σ N+1 vector size and resulting in an alternate complexity of O(σ² M N²). It should be noted that implementing this version is harder to code, and the gain is significant only when σ<<1. Note further that the gradient must be computed a number of times (typically 10-20 times) for the parameters to converge to a local maxima.

11. Global Search Algorithm: Given a sample hypothesis H, the local search method described in the preceding section can be used to search for the optimal solution in the parameter space. However, it should be stressed that the prior distribution Pr[D|H] is multimodal and therefore the local search based on the gradients by itself cannot evaluate the best value of the parameters. Instead, one must rely on a sampling of the parameter space to find points that are likely to be near the global maximum. In this respect, examination of the parameter space indicates that the parameters corresponding to the number and locations of restriction sites present the largest amount of multimodal variability. Therefore, for purposes of optimization, the sampling may be restricted to a sub-space of the original parameter space. In a specific embodiment of the present invention, the following sampling is used:

\overline{h}=(N; h₁, h₂, . . . , h_{N}).

In this embodiment, the conditional observation probability density Pr[D|H] can be evaluated pointwise in time 0(σ M N), and the nearest local maxima located in time 0(σM N²).

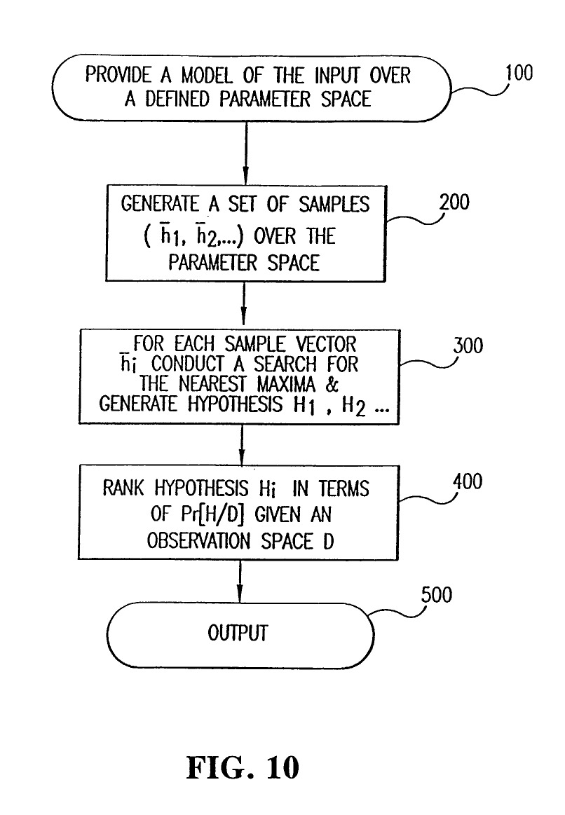

More specifically, the search for an optimal solution in a preferred embodiment of the present invention proceeds as follows, the method being illustrated in FIG. 10.

At 100, provide a model of the input signal over a defined parameter space (see preceding sections).

At 200, the method proceeds with generating a set of samples (\overline{h₁}, \overline{h₂}, \overline{h₃}, . . . ) of the parameter space, where \overline{h₁} is defined as above. The selection of the sample set is described below.

Next, at 300, these sample points are then used to begin a local search for the nearest maxima and provide hypotheses (H₁, H₂, H₃, . . . ) that correspond to the set of samples in block 200. The local search is performed using a gradient search as described previously, the computation of which is performed efficiently using dynamic programming.

Finally, at step 400, the generated hypotheses Hᵢ are ranked in terms of their posterior probability density Pr[H|D] (whose relative values also lead to the quality measure for each hypothesis), and one or more hypotheses (leading to maximal posterior probability density) or otherwise estimated to be optimal are provided to output 500 as the final answer.

This section focuses on the implementation of block 200. It should be noted first that even after restricting the sampling space, the high dimension of the space makes the sampling task daunting. Even if the space is discretized (for instance, each hᵢ∈{0, 1/200, . . . , j/200, . . . , 1}, there are still far too many sample points

$$( \begin{matrix}200 \\ N\end{matrix} )$$

even for a small number of cuts (say, N=8). However, in accordance with a specific embodiment of the present invention, the efficiency of the computation can be improved if an approximate solution is acceptable. To this end, in accordance with the present invention, the following two approaches (and their combination) are used:

(1) Approximated Bayesian probability densities can be used in conjunction with a branch and bound algorithm to reject a large fraction of the samples without further local analysis.

(2) An approximated posterior distribution for the location of the cut sites can be used in conjunction with a Monte Carlo approach to generate samples that are more likely to succeed in the local analysis.

In a preferred embodiment, the two methods can be combined. For instance, the first approach can be used to generate the best (one or more) hypothesis with a given small number of cuts (say, 5). The generated hypothesis can next be used to improve the approximated posterior probability to be used in the second approach. Note also that, if the data quality is “good,” rather simple versions of the heuristics (for global search) lead to algorithms that yield good results quite fast. Following is a description of both approaches used in accordance with the present invention.

Initially, in a preferred embodiment, the parameter N is searched in strictly ascending order. This means one first evaluates the (single) map with no cuts, then applies a global search and a gradient search to locate the best map with 1 cut, then the best map with 2 cuts, etc. One continues until the score of the best map of N cuts is significantly worse than the best map of 0 . . . N−1 cuts.

12. Approximating Bayesian Probability Densities: In a preferred embodiment of the present invention, the global search for a particular N uses an approximate Bayesian probability density with a scoring function that is amenable to efficient branch-and-bound search. Good scores for a given molecule Dⱼ essentially requires that as many cut locations s₁ⱼ, . . . , s_{Mj,j} as possible must line up close to h₁, h₂, . . . , h_{N} in one of the two orientations (left-to-right vs. right-to-left. This means that any subset of the true map h₁, h₂, . . . , hₘ (m<N) will score better than most other maps of size m, assuming that the digest rate is equal (p_{c}=p_{c1}= . . . =p_{cN}). For physical reasons, the variation among the digest rates is quite small, thereby justifying the above assumption and explicitly permitting these two parameters to be the same. For example, if (h₁, h₂, . . . , h_{N}) is the correct map, one expects maps with single cuts located at [hᵢ] (1≦i≦N) to score about equally well in terms of the Bayesian probability density. Similarly, maps with two cuts located at pairs of [hᵢ, hⱼ] (1≦i<j≦N) score about equally well and better than two cut maps chosen arbitrarily. Furthermore, the pair-cut probability densities are more robust than the single cut probability densities with respect to the presence of false cuts, hence, less likely to score maps with cuts in other than the correct locations.

An approximate score function used for a map (h₁, h₂, . . . , h_{N}) is the smallest probability density for any pair map [hᵢ, hⱼ], which is a subset of (h₁, h₂, . . . , h_{N}). In a preferred embodiment, these pair map probability densities are precomputed for every possible pair ([hᵢ, hⱼ]), if hᵢ, hⱼ are forced to have only K values along some finite sized grid, for example at exact multiples of ½% of the total molecule length for K=200. The pair map probability densities can be expressed in the form of a complete undirected graph, with K nodes corresponding to possible locations, and each edge between node “i” to “j” having an edge value equal to the precomputed pair map probability density of [hᵢ, hⱼ]. A candidate map (h₁, h₂, . . . , h_{N}) corresponds to a clique of size N in the graph, and its approximate score corresponds to the smallest edge weight in the clique.

In general, the clique problem (for instance, with binary edge weights) is NP-complete and may not result in any asymptotic speedup over the exhaustive search. For this problem, however, effective branch-and-bound search heuristics is devised in a preferred embodiment.

Consider first the problem of finding just the best clique. In accordance with the present invention, several heuristic bounds can be used to eliminate much of the search space for the best clique. In a specific embodiment, the following two are used:

(1) The score of any edge of a clique is an upper bound on the score of that clique. If the previous best clique found during a search has a better (higher) score than the score of some edge, all cliques that include this edge can be ruled out;

(2) For each node in the graph, precompute the score of the best edge that includes this node. If the previous best clique found during a search has a better (higher) score than this node score, all cliques that include this node are ruled out.

As with all branch-and-bound heuristics, the effectiveness of these techniques depends on quickly finding some good solutions, in this case cliques with good scores. Experimentally, it was found that an effective approach to be used in this problem is to sort all K nodes by the Bayesian scores of the corresponding single cut map. In other words, in a preferred embodiment, the method first tries nodes that correspond to restriction site locations that have a high observed cut rate in some orientation of the molecules. Also, the nodes corresponding to cut sites of the best overall map so far (with fewer than N cut sites) are tried first.

For data consisting of a few hundred molecules, the branch-and-bound heuristics allows exhaustive search in under a minute on a Sparc System 20 with N≦7 (with K=200). For N>7, a simple step-wise search procedure that searches for the best map (h₁, h₂, . . . , h_{N}) by fixing N−7 nodes based on the previous best map, works well. The N−7 nodes selected are the optimal with respect to a simple metric, for instance, the nodes with the smallest standard error (i.e., ratio of standard deviation to square root of sample size).

Next, the global search is modified to save the best B (typically 8000) cliques of each size and then the exact Bayesian probability density is evaluated at each of these B locations, adding certain reasonable values for parameters other than (N; h₁, . . . , h_{N}). In a preferred embodiment, these parameters can be taken from the previous best map, or by using some prior values if no previous best map is available. For some best scoring subset (typically 32—64) of these maps, a gradient search is used in a specific embodiment to locate the nearest maxima (and also to provide accurate estimates for all parameters), and the best scoring maxima are used as the final estimate for the global maxima for the current value of N.

Several variations to the global search described here, can be used in alternate embodiments. For example, it was found that for large values of N, the approximate score diverges from the true Bayesian score. To reduce the reliance on the approximate score, the step-wise search procedure can be modified to fixing, for example, N−3 nodes from the previous best map instead of N−7. For the same value of B, this modification increases the fraction of the search space that is scored with the exact Bayesian score. Fixing N−1 or even N−2 nodes would allow essentially the entire remaining search space to be scored with the exact Bayesian score in alternative embodiments. It should be noted that a potential drawback of this modified embodiment is that the amount of backtracking has been reduced and hence a wrong cut site found for small N is harder to back out of.

Additionally, instead of searching the space in strictly ascending order of N, in an alternative embodiment, it is quicker to use a “greedy” search to locate a good map for a small value of N, for example 5, and then use the more exhaustive search with backtracking to extend it to larger values of N. For a large number of cuts (as in BACS), this heuristic leads to significant computational savings, because the molecule orientations are known (with high probability) once the best map with N=5 is found. With known molecule orientations, even a greedy search using exact Bayesian scores can locate the correct map with high probability. The final, more exhaustive search, is needed in a specific implementation mainly to get a good quality measure of the result. Further, to fix the N−2 or N−3 best nodes it might be better to use a greedy search with exact Bayesian scores. In this approach, cuts are deleted successively, one cut at a time, and the cut which reduces the exact Bayesian score the least is identified.

12. A Quality Measure for the Best Map: In accordance with a preferred embodiment, a quality measure for the best map obtained using the present invention is provided by the ratio of the estimated probability of the dominant mode of the posterior probability density Pr[H|D] to the probability of the sum of values computed for the N best peaks of the multi-modal Pr[H|D]. See also FIG. 1-block 40, and FIG. 10, block 400. Thus, in a preferred embodiment, the cost function is a constant multiple of the posterior probability density, and is not normalized by dividing it by the integral of the cost over the entire parameter space.

Specifically, the probability of the dominant mode of the posterior probability density is computed in a preferred embodiment by integrating the probability density over a small neighborhood of the peak (computed in the parameter space). Next, the following simplifying assumption is made: All peaks of the posterior probability density are sharp and the integral of the cost function over a neighborhood where the cost value is larger than a specific value is proportional to the peak density. Thus, in accordance with the present invention, if the N most dominant peaks of the posterior probability density are known, the cost over the entire parameter space can be approximated by the integral over the N neighborhoods of these peaks, where typically N=64.

This quality of goodness measure simplifies the computation considerably, while producing a very good estimate. To take into account sampling errors, such as those which occur when the number of molecules is small, the density of the best map can be reduced by an estimate of the sampling error. This approach tends to make the computed quality measure somewhat pessimistic. However, the reduction provides a lower bound.

It should be noted that the approach of generating a set of restriction maps with different “quality measures” has the additional benefit that this information can be used to safeguard the database from being corrupted and to provide very important feedback to the experimenters who could repeat their experiment and gather more data when the estimated qualities are too low.

In addition, as noted above, the output of the algorithm used in accordance with the present invention is guaranteed to have the optimal accuracy. The demand for this high accuracy is justified by the fact that even a small loss of accuracy contributes to an exponential growth in the complexity of the “contig” problem.

Finally, it is important to note that the method of the present invention described in the preceding sections generalizes easily to other cases where the data model differs significantly. For instance, with BAC data one can expect the end-fragments to occasionally break and to miss the interior fragments occasionally. Other important situations involve the models for circular (non-linearized) DNA, genomic (uncloned) DNA, and data sets consisting of clones of two or more DNA's. Other situations involves augmentation with some more (helpful) data that can be made available by appropriate changes to the chemistry, e.g., the presence of external standards allowing one to work with absolute fragment sizes, or external labeling disambiguating the orientation or alerting one to the absence of a fragment. The flexibility of the approach derives from its generality and cannot be achieved by the simpler heuristics.

### EXAMPLES

The following Examples are included solely to provide a more complete and consistent understanding of the invention disclosed and claimed herein. The Examples do not limit the scope of the invention in any fashion.

Cell Growth and DNA Preparation:

The E. coli 0157:H7 strain used for the mapping of this organism was the same strain used for sequencing (Perna et al. 2001). E. coli 0157:H7 was grown to late log phase in LB broth (per liter: 10 g tryptone, 5 g yeast estract, 5 g NaCl). Bacteria were washed in TNE buffer (10 mM Tris, pH 7.2, 200 mM NaCl, 100 mM EDTA) and embedded in low-melting, 1% agarose gel (“InCert”-brand, FMC Corporation, Philadelphia, Pa.) to form 20 μl inserts. Bacteria were lysed with lysozome (1 mg/mL) followed by proteinase K treatment (0.5 mg/mL) in buffer containing EDTA (100 mM, pH 8.0), sodium deoxycholate (0.2%), Brij-58 (polyoxyethylene 20 cetyl ether, 0.5%), and sarcosyl (0.5%). Prior to use, the DNS inserts were washed thoroughly overnight in TE to remove excess EDTA. To extract DNA, washed inserts were melted at 72° C. for 7 min. A β-agarase solution (100 μl of TE+1 μl (1 U) β-agarase, New England Biolabs, Beverly, Mass.), prewarmed to 40° C., was added to the melted inserts, and allowed to incubate at 40° C. for 2 h. This concentrated DNA sample was equilibrated to room temperature. Then 10 μl of the DNA sample was added to 490 μl of 30 pg/μl lambda bacteriophage DNA (New England Biolabs). The samples were mounted onto an optical mapping surface as described herein and examined under a fluorescence microscope to check the integrity of the DNA sample, and also to check the concentration of the genomic DNA. If further dilution was needed, 100 μl of 30 pg/μl lambda bacteriophage was added to the sample. The sample was again examined under the microscope. Dilution and examination was iterated until the genomic DNA was dilute enough so that only a few genomic molecules could be seen distinctively in each field of view of the microscope.

Surface Preparation and Calibration:

Glass cover slips (18×18 mm; “Fisher's Finest,” Fisher Lab Products, Pittsburgh, Pa.) were racked in custom-made Teflon racks, and cleaned by boiling in concentrated nitric acid for at least 12 h. The cover slips were rinsed extensively with high-purity, dust-free water until the effluent attained natural pH. The cleaning procedure was repeated with concentrated hydrochloric acid, which hydrolyzes the glass surface, preparing it for subsequent derivatization. The cleaned cover slips were rinsed extensively, and any unused cover slips were stored at room temperature under ethanol in polypropylene containers.

A stock (2% by weight) solution of 3-aminopropyldiethoxymethylsilane (APDEMS; Gelest, Morrisville, Pa.), distilled under argon, was prepared by dissolving APDEMS in deionized water and allowed to hydrolyze on a shaker at room temperature for 7.5 h. Thirty-six cleaned cover slips were treated in 4.2 to 5.8 μm hydrolyzed APDEMS in 250 mL distilled ethanol on a 50 rpm shaker at room temperature for 48 h. Any unused derivatized surfaces were stored in the silane solution and were used for up to two weeks. The surfaces were assayed by digesting lambda bacteriophage DNA with 60 unites of XhoI enzyme diluted in 100 μl of digestion buffer with 0.02% Triton at 37° C. to determine optimal digestion times, which ranges from 9 to 12 min.

Sample Mounting:

Capillary action was used to draw DNA solution (5 μl E. coli O157:H7) between a derivatized surface and a glass slide. Two sets of protocols were used for digestion: NheI: the resulting sandwich was allowed to sit at room temperature for a few minutes, then carefully peeled from the slide. Surface-mounted DNA was digested with 1.5 μL NEB buffer 2 for 8-15 min. at 37° C., in a humidity chamber. The buffer was aspirated from the surface to halt digestion, followed by washing (2×) with high-purity water. The mounted sample was dried on a 55° C. heating block for one minute. XhoI: surface-mounted DNA was digested with 3.0 μL (60 U) XhoI (New England Biolabs) in 100 μL of 1×NEB Buffer 2 for 9-12 min. in a humidity chamber at 37° C. The enzyme solution was carefully pipetted from the surface, and the surface was washed (2×) with excess filtered, high-purity water. The surface was thoroughly dried in a dehumidfying chamber using dessicant (Drierite).

Image Acquisition:

Mounted DNA molecules were stained by placing 5 μL 0.1 μM YOYO-1 (in TE containing 20% β-mercaptoethanol; Molecular Probes, Eugene, Oreg.) on a clean slide. The mounted sample was carefully placed on top of the YOYO-1 solution, avoiding air bubbles. Consecutive microscope images were semiautomatically collected under software control (GenCol software; Lai et al. 1999; Lin et al. 1999b) using 63×microscope objectives. Co-mounted lambda DNA molecules were used to estimate the rate of digestion and to provide a fluorescence standard for sizing (Jing et al., 1999, Genome Res. 9:175-181; Lai et al., 1999, Nat. Genet. 23:309-313; and Lin et al., 1999, Science 285:1558-1562).

Image Processing:

Images were processed using new software for semiautomatic processing, Semi-Autovis. Fine editing of molecule markups was performed using an image editing program, Visionade (Aston et al., 1999b, Methods Enzymol. 303:55-73). Semi-Autovis calculates restriction maps of molecules from an overlapping set of images. User input is limited to identification of the approximate location of suitable molecules, a step that will be automated in future versions of the software. Semi-Autovis then locates the exact location of the center line (backbone) of all selected molecules, as well as: any other molecules that are nearby, the most likely locations of the restrictions sites on each molecule based on the variation in intensity, and the integrated intensity of each molecule fragment so identified. This is done on each image separately. The results from overlapping images are then combined to merge long molecules, and sizes are translated from intensity units to an absolute scale (kilobases) by identifying nearby size-standard molecules in the image whose restriction map and size are known. This produces a physical restriction map for each molecule identified by the user.

A critical feature of Semi-Autovis is that it can automatically deal with crossing molecules, bright spots near molecules, and other object imperfections that can interfere with accurate fragment calling and sizing. Visionade required manual editing to eliminate object noise. Semi-Autovis, in contrast, identifies DNA molecules by looking for long, thin, bright objects that vary slowly in orientation. In the first phase, an algorithm identifies these isolated regions in the image, using both the fluorescence intensity and local directionality properties at each pixel. This is done by first applying a pattern matching filter in the shape of an idealized molecule, which is convolved with the input image in 16 different orientations and produces 16 new images. Each image corresponds to one of 16 different directions, and the value of a pixel in one of these images represents a calculation of the degree to which the pixel appears to lie on a molecule in the particular direction. An image is then constructed which contains, at each pixel, the highest of the 16 values for that pixel. These images are subjected to a threshold filter to remove both the background and small bright objects that do not match molecules in shape. This operation dramatically reduces the number of pixels that remain to be processed. The remaining pixels are clustered into connected regions, each of which may contain one or more DNA fragments. The filter tends to include pixels corresponding to small gaps between fragments, whether in the same molecule or different nearby molecules.

In the next phase, Semi-Autovis identifies the “backbones” (or center-lines) of the DNA fragments by computing the intensity contours at various levels of intensity and identifying “pointed ends” on these contours. The set of all pointed ends represents the end points of fragments filtered at thresholds of various levels. Collectively the set defines the center lines of the DNA fragments. This formulation has the advantage of only assuming that all objects are thin, without requiring them to be totally straight, and allowing multiple objects to cross each other. In addition, the locus of the thresholded fragment end points can be computed efficiently.

The backbones (DNA center lines) must now be processed to separate out crossed DNA molecules and locate gaps in the DNA molecules corresponding to restriction sites. First, each point on the backbone with more than two continuations (a crossing point) is analyzed by computing the angles of each backbone segment incident at that point and matching backbone segments lying in approximately the same direction. Next, each pair of matched up segments are joined into one DNA molecule. Any unmatched segments at the crossing point are treated as molecule ends. Now each molecule is defined by one or more backbone lines (possibly curved), where each line corresponds to one or more fragments. Within each backbone line the gaps between fragments will be small, because larger gaps would break up the DNA molecule into separate backbone lines. The next step is to locate the smaller gaps by analyzing the intensity profile along the backbone lines. A smooth intensity signal along the backbone is computed; for each position along the backbone, the intensity is calculated by summing the intensities for a set of pixels which are close to the backbone and lying along a line orthogonal to the backbone at that position.

Gaps are characterized by intensity dips with a characteristic inverted Gaussian shape. The parameters that characterize gaps are derived empirically from hand-marked-up training sets, and the final parameter set is able to find over 95% of the gaps that a human was able to identify with −4% false positives, versus 2.5% for human markups (data not shown).

The backbone section corresponding to each fragment is used to define an area roughly three times as wide as the actual molecule. If two areas overlap, pixels are assigned based on the nearest backbone pixel. The intensity of each fragment's area is integrated and used as an estimate of the mass of the fragment, which is later normalized.

Map Construction:

Another software package called Gentig (Anantharaman et al., 1998, Courant Technical Report 760 Courant Institute, New York University, New York; Anantharaman et al., 1999, The Seventh International conference on Intelligent Systems for Molecular Biology, 7:18-27; and Lai et al. 1999, supra) takes these single molecule restriction maps and combines them into a genome-wide contig using the Bayesian data error model described hereinabove. This model simultaneously estimates the data error rated while generating a contig map with as little error as possible by using all data redundancy present in the overlapping single-molecule maps. Gentig computes a false positive probability each time a map overlap is considered, and accepts the resulting contig only when it is quite sure that the overlap could not be due to chance given the data errors. In this fashion, Gentig avoids the exponential cost of the backtracking that this problem requires to ensure the best possible contig. This does mean that occasionally the software will fail to close a gap in the contig when the quantity of data is barely sufficient in theory, but only a very small fraction of extra data is sufficient to allow Gentig to close the gap without exponential backtracking.

The results of this example are depicted in the optical mapping surface shown in FIG. 2A, the resulting optical contig map depicted in FIG. 2B, the optical map fragment size graph depicted in FIG. 3, and the consensus optical map shown in FIG. 4, all described in greater detail hereinabove. Taken collectively, these results indicate that the present approach can be used to generate optical contig maps that correlate well with genome sequence results obtained via conventional means. The results also show that the optical contig maps constructed according to the present invention are highly useful to verify, refute, or access the accuracy of a known gene or genomic sequence obtained by other means.

What is claimed is:

1. A method of validating or refuting a known nucleic acid sequence, the method comprising:
(a) elongating and fixing a plurality of nucleic acid molecules along their length onto a solid planar surface so that the nucleic acid molecules are individually analyzable and accessible for enzymatic reactions; and
(b) reacting the nucleic acid molecules from step (a) with a restriction endonuclease under conditions and for a time wherein the restriction endonuclease cleaves the nucleic acid molecules at a plurality of sequence-specific positions, thereby yielding a plurality of cleaved fragments of discernable length fixed to the surface; then
(c) discerning the lengths of the cleaved fragments generated in step (b) and then
(d) from the lengths discerned in step (c), assembling an optical contig map; and then
(e) comparing the optical contig map from step (d) to the known nucleic acid sequence, whereby the known nucleic acid sequence is validated or refuted.

2. The method of claim 1, wherein in step (a), the nucleic acid molecules are elongated and fixed onto a substrate having a positive charge density.

3. The method of claim 1, wherein in step (a), the nucleic acid molecules are elongated and fixed onto a derivatized glass substrate.

4. The method of claim 3, wherein the glass substrate is derivatized to have a positive charge density.

5. The method of claim 3, wherein the glass substrate is derivatized with an amino group-containing agent.

6. The method of claim 3, wherein the glass substrate is derivatized with 3-aminopropyltriethoxysilane, 3-methylaminosilane, or poly(lysine).

7. The method of claim 1, wherein in step (c), the lengths of the cleaved fragments are discerned using optical means for visualizing cleavage of the nucleic acid molecules.

8. The method of claim 7, wherein the optical means for visualizing cleavage of the nucleic acid molecules comprises an optical microscope.

9. The method of claim 7, wherein the optical means for visualizing cleavage of the nucleic acids comprises means for staining the nucleic acid molecules, and a microscope capable of detecting the staining means.

10. The method of claim 1, wherein in step (a), the plurality of nucleic acid molecules are genomic DNA molecules comprising an entire genome of an organism.

11. The method of claim 10, wherein the genomic DNA molecules comprise the entire genome of a bacteria.

12. The method of claim 10, wherein the genomic DNA molecules comprise the entire genome of an E. coli.

13. The method of claim 10, wherein the genomic DNA molecules comprise the entire genome of an E. coli, type O157:H7, strain EDL933.

14. A method of assessing the accuracy of a known genome-wide nucleic acid sequence of an organism, the method comprising:
(a) to individual genomic DNA molecules comprising an entire genome of the organism, the DNA molecules being elongated and fixed along their length onto a solid planar surface so that the nucleic acid molecules are individually analyzable and accessible for enzymatic reactions, reacting the DNA molecules with at least one restriction endonuclease under conditions and for a time wherein the restriction endonuclease cleaves the DNA molecules at a plurality of sequence-specific positions, thereby yielding a plurality of cleaved fragments of discernable length fixed to the surface; then
(b) discerning the lengths of the cleaved fragments generated in step (a) and then
(c) from the lengths discerned in step (b), assembling an optical contig map; and then
(d) comparing the optical contig map from step (c) to the known, genome-wide nucleic acid sequence, whereby the accuracy of the known nucleic acid sequence is assessed.

15. The method of claim 14, wherein step (a) is reiterated two or more times, using a different restriction endonuclease for each reiteration; and steps (b) and (c) are repeated for each reiteration, whereby a corresponding optical contig map is generated for each reiteration, and then further comprising, after step (c) and prior to step (d): (c)(i) compiling the optical contig maps into a unified consensus optical contig map: and wherein in step (d), the unified consensus optical contig map from step (c)(i) is compared to the known, genome wide nucleic acid sequence.

16. The method of claim 14, wherein in step (a), the individual genomic DNA molecules are elongated and fixed onto a substrate having a positive charge density.

17. The method of claim 14, wherein in step (a), the individual genomic DNA molecules are elongated and fixed onto a derivatized glass substrate.

18. The method of claim 17, wherein the glass substrate is derivatized to have a positive charge density.

19. The method of claim 17, wherein the glass substrate is derivatized with an amino group-containing agent.

20. The method of claim 17, wherein the glass substrate is derivatized with 3-aminopropyltriethoxysilane, 3-methylaminosilane, or poly(lysine).

21. The method of claim 14, wherein in step (b), the lengths of the cleaved fragments are discerned using optical means for visualizing cleavage of the nucleic acid molecules.

22. The method of claim 21, wherein the optical means for visualizing cleavage of the nucleic acid molecules comprises an optical microscope.

23. The method of claim 21, wherein the optical means for visualizing cleavage of the nucleic acids comprises means for staining the nucleic acid molecules, and a microscope capable of detecting the staining means.

24. The method of claim 14, wherein the genomic DNA molecules comprise the entire genome of a bacteria.

25. The method of claim 24, wherein the genomic DNA molecules comprise the entire genome of an E. coli.

26. The method of claim 24, wherein the genomic DNA molecules comprise the entire genome of an E. coli, type O157:H7, strain EDL933.

27. A consensus optical contig map of an entire genome of an organism, the map constructed by a series of steps comprising:
(a) extracting genomic DNA molecules from the organism; and then
(b) elongating and fixing a plurality of the genomic DNA molecules from step (a) along their length onto a solid planar surface so that the DNA molecules are individually analyzable and accessible for enzymatic reactions; and
(c) reacting the DNA molecules from step (b) with at least one restriction endonuclease under conditions and for a time wherein the restriction endonuclease cleaves the DNA molecules at a plurality of sequence-specific positions, thereby yielding a plurality of cleaved fragments of discernable length fixed to the surface; then
(d) discerning the lengths of the cleaved fragments generated in step (c) and then
(e) from the lengths discerned in step (d), assembling the consensus optical contig map.

28. The map of claim 27, wherein steps (b) and (c) are reiterated two or more times, using a different restriction endonuclease for each reiteration; and step (d) is repeated for each reiteration, whereby a corresponding optical contig map is generated for each reiteration, and then further comprising, in step (e) compiling the optical contig maps into a unified consensus optical contig map.

29. The map of claim 27, wherein in step (b), the individual genomic DNA molecules are elongated and fixed onto a substrate having a positive charge density.

30. The map of claim 27, wherein in step (b), the individual genomic DNA molecules are elongated and fixed onto a derivatized glass substrate.

31. The map of claim 30, wherein the glass substrate is derivatized to have a positive charge density.

32. The map of claim 30, wherein the glass substrate is derivatized with an amino group-containing agent.

33. The map of claim 30, wherein the glass substrate is derivatized with 3-aminopropyltriethoxysilane, 3-methylaminosilane, or poly(lysine).

34. The map of claim 27, wherein in step (d), the lengths of the cleaved fragments are discerned using optical means for visualizing cleavage of the nucleic acid molecules.

35. The map of claim 34, wherein the optical means for visualizing cleavage of the nucleic acid molecules comprises an optical microscope.

36. The map of claim 34, wherein the optical means for visualizing cleavage of the nucleic acids comprises means for staining the nucleic acid molecules, and a microscope capable of detecting the staining means.

37. The map of claim 27, wherein the genomic DNA molecules comprise the entire genome of a bacteria.

38. The map of claim 37, wherein the genomic DNA molecules comprise the entire genome of an E. coli.

39. The map of claim 37, wherein the genomic DNA molecules comprise the entire genome of an E. coli, type O157:H7, strain EDL933.
